SEQUENCE LISTING (6 sequences)

SEQ ID NO: 1 - RNA, 100 nt, Artificial sequence
  Sequence of the sgRNA targeting the iGFP
guuuccggcuagggauaacaguuuuagagcuagaaauagcaaguuaaaauaaggcuaguccguuaucaacuugaaaaaguggcaccgagucggugcuuuu

SEQ ID NO: 2 - RNA, 312 nt, Artificial sequence
  RNA donor sequence (312 nt) to repair the stop codons in the iGFP
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgac

SEQ ID NO: 3 - DNA, 720 bp, Artificial sequence
  DNA sequence of the iGFP
atggtgagcaagggcgaggagctgttcaccggggtggtgcccatcctggtcgagctggacggcgacgtaaacggccacaagttcagcgtttccggctagggataacagggtaatacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 4 - DNA, 683 bp, Artificial sequence
  CasQ2 DNA Donor with U6 promotor sequence
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagacgggatcctgactcattctatagaataatcctataattgagggtgagcaccaaatgtcctaagaagtcctcgtggaaaaatcacactctgctctccacattagaagctgtgtatgtgcaggggttactcaactctcttgaatcctgtttcagatggctacgaattcctggagatcctgaaacaggttgcccgggacaatactgataatcctgacttgtctattttatggattgatcctgacgactttcctctggtgagtggctccagccagcgccccaccctcagttgctgtcacaacctcatgattgcgttcagcatctgaggaggtgaaataggcaggggcacttgaagaaacccagtggcggtcctcacccagtgtgggaggtcttggcccttggggcagtattgaacttggggcctggtgctgaccctgaaagg

SEQ ID NO: 5 - DNA, 430 bp, Artificial sequence
  CasQ2 DNA Donor without U6 promotor sequence
tgactcattctatagaataatcctataattgagggtgagcaccaaatgtcctaagaagtcctcgtggaaaaatcacactctgctctccacattagaagctgtgtatgtgcaggggttactcaactctcttgaatcctgtttcagatggctacgaattcctggagatcctgaaacaggttgcccgggacaatactgataatcctgacttgtctattttatggattgatcctgacgactttcctctggtgagtggctccagccagcgccccaccctcagttgctgtcacaacctcatgattgcgttcagcatctgaggaggtgaaataggcaggggcacttgaagaaacccagtggcggtcctcacccagtgtgggaggtcttggcccttggggcagtattgaacttggggcctggtgctgaccctgaaagg

SEQ ID NO: 6 - DNA, 20 bp, Artificial sequence
  CASQ2 gRNA
accccgatctgagcatcctg

(19) United States
(12) Patent Application Publication
(10) Pub. No.: US 2020/0123542 A1
(43) Pub. Date: Apr. 23, 2020

(54) RNA COMPOSITIONS FOR GENOME EDITING

(71) Applicant: EmendoBio Inc., Wilmington, DE (US)

(72) Inventors: David Baram, Tel Aviv (IL); Lior Izhar, Tel Aviv (IL); Rafi Emmanuel, Ramla (IL)

(73) Assignee: EmendoBio Inc., Wilmington, DE (US)

(21) Appl. No.: 16/703,766

(22) Filed: Dec. 4, 2019

Related U.S. Application Data
(63) Continuation of application No. 16/341,835, filed on Apr. 12, 2019.
(60) Provisional application No. 62/480,954, filed on Apr. 3, 2017.
(60) Provisional application No. 62/452,222, filed on Jan. 30, 2017.
(60) Provisional application No. 62/435,270, filed on Dec. 16, 2016.
(60) Provisional application No. 62/425,520, filed on Nov. 22, 2016.
(60) Provisional application No. 62/411,328, filed on Oct. 21, 2016.
(60) Provisional application No. 62/408,203, filed on Oct. 14, 2016.

(51) Int. Cl. C12N 15/11; C12N 15/113; C12N 9/22; A61K 38/46; A61K 31/713; A61K 31/7088

(52) U.S. Cl. CPC C12N 15/11; C12N 15/113; C12N 9/22; C12N 2310/20; A61K 31/713; A61K 31/7088; C12N 2800/80; A61K 38/465

(57) ABSTRACT

RNA is a preferred composition for delivering genes to target cells for inducing genome editing. While RNA-guided DNA nucleases and their guide-RNA molecules can be easily delivered to a cell as RNA, a donor template is normally delivered as DNA for homologous recombination mediated repair in the genome following a double strand break. It is an object of the present invention to provide a RNA donor template for inducing gene correction following a double strand break.

This application is a continuation of U.S. application Ser. No. 16/341,835, filed Apr. 12, 2019, which is a § 371 national stage of PCT International Application No. PCT/US2017/056332, filed Oct. 12, 2017 and claiming the benefit of U.S. Provisional Application Nos. 62/480,954, filed Apr. 3, 2017; 62/452,222, filed Jan. 30, 2017; 62/435,270, filed Dec. 16, 2016; 62/425,520, filed Nov. 22, 2016; 62/411,328, filed Oct. 21, 2016; and 62/408,203, filed Oct. 14, 2016, the contents of each of which are hereby incorporated by reference.

Throughout this application, various publications are referenced, including referenced in parenthesis. The disclosures of all publications mentioned in this application in their entireties are hereby incorporated by reference into this application in order to more fully describe the state of the art to which this invention pertains.

This application incorporates-by-reference nucleotide and/or amino acid sequences which are present in the file named “191204_89069-AA-PCT-US_SequenceListing_DH.txt”, which is 4.41 kilobytes in size, and which was created Dec. 3, 2019 in the IBM-PC machine format, having an operating system compatibility with MS-Windows, which is contained in the text file filed Dec. 4, 2019 as part of this application.

### BACKGROUND

Targeted genome modification is a powerful tool that can be used to reverse the effect of pathogenic genetic variations and therefore has the potential to provide new therapies for human genetic diseases. Current genome engineering tools, including engineered zinc finger nucleases (ZFNs), transcription activator-like effector nucleases (TALENs), and most recently RNA-guided DNA endonucleases such as CRISPR/Cas nucleases and orthologues thereof, produce sequence-specific DNA breaks in a genome. The modification of the genomic sequence occurs at the next step and is the product of the activity of cellular DNA repair mechanisms triggered in response to the newly formed DNA break.

The present invention provides compositions and methods for a safe and efficient induction of double strand break for gene editing using RNA compositions. The RNA Exhibit B compositions and methods described herein are useful in improving the efficiency and safety of gene editing.

### SUMMARY OF THE INVENTION

The present invention recites a donor RNA template having homology arms to the target gene, or more generally any DNA site, and at least one insert sequence between the homology arms. The donor RNA template, also referred to herein as a “RNA template,” “RNA-based donor,” “RNA donor,” or more simply “donor,” or “template,” is useful for gene editing applications. Specifically, the RNA template contains at least one insert having a sequence difference relative to the DNA target site, such that the sequence difference is an alteration intended to be introduced into the target DNA site sequence. Accordingly, the sequence information of the RNA template replaces the original sequence of the DNA target site.

A homology arm can be 1, 2, 3, 4, 5, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000 bases long and more. A homology arm may have the same or a different length relative to other homology arms of the RNA-based donor. Each possibility represents a separate embodiment of the present invention.

In an embodiment, each of the homology arms varies in length. In a non-limiting example, a homology arm downstream of the insert is longer than a homology arm upstream of the insert. In another non-limiting example, a homology arm upstream of the insert is longer than a homology arm downstream of the insert.

The insert can be 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1000 or more. The homology arms are designed to anneal or hybridize to the genomic target DNA sequences that flank the intended DSB site in the target DNA.

Any difference in sequence between the RNA-based donor and target DNA, either in the number of nucleotide bases or nucleotide base identity, is considered part of an insert sequence, which ultimately replaces the original DNA target sequence during RNA-templated DNA repair. Thus, for the purposes of genome editing, a RNA-based donor may be designed to contain at least one difference in sequence relative to the target DNA sequence, such that the at least one sequence difference is an alteration intended to be introduced into the target DNA site sequence upon RNA-templated DNA repair. Such alterations include, but are not limited to, introducing an additional new sequence into the original DNA target sequence, deleting a portion of the original DNA target sequence, altering the sequence identity of one or more nucleotides in the original DNA target sequence, or any combination of the above.

In an embodiment, the RNA donor template of the present invention is a ssRNA.

In an embodiment, the RNA donor template contains a self-annealing RNA segment located on at least one of its termini. The self-annealing RNA segment forms a RNA structure e.g. a hairpin loop at the 5′, 3′ or both ends of the RNA donor template.

In an embodiment, the RNA donor template is devoid of a methylated cap at its 5′ termini. In an embodiment, the RNA donor template is a non-naturally occurring RNA.

In an embodiment, the RNA donor of the present invention is fused/linked to a DNA segment on at least one of its ends. In an embodiment, the RNA donor of the present invention is covalently linked to a DNA segment. In an embodiment, the RNA donor of the present invention is linked to a DNA segment by base pairing of complementary nucleotide bases.

In an embodiment, the DNA segment fused to the RNA donor is 5, 10, 15, 20, 30, 40, 50, 100, 250 bases or more in length.

In an embodiment, the RNA donor of the present invention is fused on its 3′ end, 5′ end, or both, to a DNA segment that is homologous to the target genomic corresponding sequence.

In an embodiment, the RNA donor of the present invention is fused in its 3′ end, 5′ end, or both, to a DNA segment that is non-homologous to the target genomic corresponding sequence.

In an embodiment, the DNA segment that is fused to at least one of the ends of the RNA donor is a hairpin structure.

In an embodiment, the DNA segment that is fused to at least one of the ends of the RNA donor is a protein binding site.

In an embodiment, the protein binding site is a restriction sequence designed to bind the binding domain of restriction enzyme that is fused to a RNA-guided nuclease according to the present invention.

In an embodiment, the protein binding site is designed to bind an endogenous protein in the cell, for example, a protein comprising a nuclear localization sequence (NLS). In an embodiment, the protein comprising a NLS is a transcription factor (TF). In an embodiment, the protein binding site is a transcription factor (TF) binding site (e.g. TBP, TAFs, Sp1, E2F, E-box, YY1, etc., including any TF binding site known in the art).

In an embodiment, the TF binding site fused to the RNA donor of the present invention is designed to bind a TF which acts on the target gene desired to be edited.

In an embodiment, the 3′ end of the RNA donor of the present invention is fused to a DNA fragment that is homologous to the target genomic corresponding sequence, and the 5′ end of the RNA donor is fused to a DNA segment that contains a TF binding site.

In an embodiment, the 5′ end of the donor RNA is fused to a cap-analog (e.g. ApppG or GpppG). Suitable cap-analogs attached to a donor RNA of the present invention include any non-methylated cap analog known in the art.

In an embodiment, the donor RNA of the present invention is at least partially modified.

In an embodiment, at least 10%, 15%, 20%, 25%, 30%, 35%, 40%, 45%, 50%, 55%, 60%, 65%, 70%, 75%, 80%, 85%, 90%, 95%, 100% of the uridine in the donor RNA of the present invention are 1-methyl pseudo-uridine or pseudo-uridine. Each possibility represents a separate embodiment of the present invention.

In an embodiment, the invention provides a ribonucleoprotein (RNP) comprising the donor RNA of the present invention associated/linked to a RNA binding protein.

In an embodiment, the RNA template of the invention is a non-naturally occurring RNA molecule.

The present invention, according to an embodiment, recites a composition for gene editing comprising a donor RNA template according to the present invention and an mRNA encoding a DNA nuclease. In an embodiment, the DNA nuclease is a RNA-guided DNA nuclease. For example, the composition for gene editing may comprise an mRNA encoding Cas9. In an embodiment, the composition further comprises a guide RNA capable of targeting the RNA-guided DNA nuclease.

The present invention, according to an embodiment, recites a composition for gene editing comprising a donor RNA template according to the present invention and a guide RNA.

The present invention, according to an embodiment, recites a composition for gene editing comprising a donor RNA template according to the present invention and at least one of:
i) a mRNA encoding RNA-guided DNA nuclease (e.g, a mRNA encoding Cas9);
ii) at least one guide RNA; and
iii) a tracrRNA.

In an embodiment, the composition of the invention comprises elements which do not naturally occurring together.

In an embodiment, a RNA template may target the same DNA strand that is targeted by a guide RNA, or the opposite DNA strand. More generally speaking, the RNA template may target the same strand or the opposite strand of a DNA that is targeted or cleaved by a nuclease, particularly in cases where the nuclease only targets or cleaves a single strand of the DNA e.g., wherein the nuclease is a nickase.

In an embodiment, the at least one of the RNAs of the composition described herein is at least partially modified. Modifications to polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of a RNA. As described herein, an example of a modified polynucleotide is a RNA containing 1-methyl pseudo-uridine or pseudo-uridine. For examples of modified polynucleotides and their uses, see U.S. Pat. No. 8,278,036: PCT International Publication No. WO 2015/006747, and Weissman and Kariko, 2015, (9): 1416-7, hereby incorporated by reference.

In an embodiment, at least one of the RNAs of the composition described herein are modified to contain 1-methyl pseudo-uridine or pseudo-uridine.

In an embodiment, the mRNA encoding the RNA guided nuclease is at least partially modified. Non-limiting examples for modifications to an mRNA of the present invention may be naturally occurring e.g., 3′-polyadenylation or 5′-capping of mRNAs. An mRNA of the present invention may capped with a methylated cap.

In an embodiment, the RNA-guided nuclease of the present invention is fused to at least one additional nucleic acid binding domain, for example, the binding domain of a restriction enzyme. Such a binding domain of a restriction enzyme is capable of binding either a DNA:DNA or DNA:RNA duplex. For example, the Cre-lox based recognition domain, or a Type II restriction enzyme binding domain, e.g. AvaII, AvrII, BanI, HaeIII, HinfI and TaqI.

In an embodiment, the donor RNA of the present invention is fused (e.g., by ligation) or associated (e.g., by base pairing) with at least one of: a guide RNA, a tracrRNA, or a guide RNA associated with or fused to a tracrRNA. In an embodiment, the donor RNA of the present invention is fused on its 3′ end, 5′ end, or both, to a guide RNA creating a RNA molecule which contains both a guide RNA and a donor template. In another embodiment, the donor RNA of the present invention is associated (e.g., by base pairing) on its 3′ end portion, 5′ end portion, or both, with a guide RNA.

In an embodiment, the donor RNA of the present invention is fused to a guide RNA with a linker, the linker being the length of 1, 2, 3, 4, 5, 10, 15, 20, 30, 40, 50, 60 bases or more, creating a RNA molecule which contains both a guide RNA and a donor template.

The RNA molecule can be further fused on its 3′ end, 5′ end, or both, to a tracrRNA also creating the duplex for binding to an effector protein (e.g., Cas9 protein). In another embodiment, the RNA molecule can be further associated (e.g., by base pairing) on its 3′ end portion, 5′ end portion, or both, with a tracrRNA. The donor RNA may be fused to or associated with a single-guide RNA (sgRNA) which activates and targets a RNA-guided DNA nuclease.

Accordingly, in an embodiment the donor RNA of the present invention is fused to the guide RNA, creating a RNA molecule comprising a guide RNA and a donor template. The RNA molecule may be fused to a tracrRNA which activates and targets a RNA-guided DNA nuclease e.g., Cas9. The donor RNA may be fused to a single-guide RNA (sgRNA) which activates and targets a RNA-guided DNA nuclease. A linker may separate a RNA donor from a guide RNA or tracrRNA, the linker being the length of 1, 2, 3, 4, 5, 10, 15, 20, 30, 40, 50, 60, 70, 80, 90, 100, 250, 500, 1000 bases or more. Thus, a single RNA molecule may contain a guide RNA portion and tracrRNA portion, which bind and guide a RNA-guided DNA nuclease to a DNA target site for cleavage, as well as a RNA donor template portion used to repair the DNA break caused by the nuclease, and optionally a linker portion(s) used to provide additional spacing between portions. Furthermore, although these portions may be fused directly to each other as described above and shown in FIG. 3A-FIG. 3E, the portions may also belong to different RNA strands and attach to each other at overlapping complementary regions via basepairing. A practical advantage of connecting portions of the donor/guide/tracr RNA molecule via basepairing is the convenience of easily adding, removing or changing the position of a portion of the donor/guide/tracr RNA molecule. Therefore, a single RNA molecule may contain a guide RNA portion, a tracr RNA portion, a RNA-based donor portion, and optionally a linker portion(s), wherein each portion is attached to another portion either by direct ligation or via basepairing, in any order. The RNA-based donor of a donor/guide/tracr RNA molecule may target the same DNA strand that is bound by the guide RNA, or the opposite DNA strand.

In an embodiment, a guide/donor RNA molecule, or donor/guide/tracr RNA molecule is fused at its 5′ end to or associated with a DNA fragment that is homologous to the target genomic corresponding sequence.

In an embodiment, the guide/donor RNA molecule, or donor/guide/tracr RNA molecule contains on at least one of its termini a self-annealing RNA segment that forms a hairpin loop. Such a terminal RNA hairpin loop increases protection from RNA degradation, thus improving the stability of the guide/donor RNA molecule, or donor/guide/tracr RNA molecule. Furthermore, the terminal RNA hairpin loop may be fused to a cap-analog (e.g. ApppG or GpppG). Alternatively, a self-annealing DNA segment may be used in place of a self-annealing RNA segment.

The presence of a RNA-donor template and the guide-RNA on a single molecule, or the association between the RNA-donor template and a guide-RNA via basepairing, facilitates transport of the donor to the nucleus and also brings the donor into close proximity to the DNA cleavage site (e.g., Cas9 target site). Other embodiments to accomplish these advantages are also envisioned, including using Cas9 fused to an additional protein domain which specifically binds the RNA donor or a DNA encoding the RNA donor.

The composition described can further comprise mRNA encoding at least one of Rad52, Rad51C, and CBS.

The composition described can further comprise a mRNA encoding a transcription activator. The transcription activator can may enhance transcription of the genomic target gene for editing.

The composition described can further comprise a mRNA encoding a nuclease, for instance, a RNA-guided DNA nuclease e.g. Cas9, Cpf1, etc.

A mRNA molecule utilized in the composition described herein may encode for variants of protein sequences, for instance, codon-optimized versions of a protein. The mRNA may also encode additional elements into a protein sequence. Such additional elements include, for example, a nuclear localization sequence (NLS) to improve import of the protein into the nuclease, degron tags, particularly cell-cycle dependent degron tags, or any epitope tag known to those of skill in the art.

The composition described can further comprise at least one RNA interference molecule such as siRNA, shRNA, miRNA and antisense RNA and dominant negative forms, designed to downregulate genes involved in or required for alternative end joining (Alt-EJ), for example PARP1 or Lig IIIa/XRCC1.

The composition described can further comprise of at least one RNA interference or silencing molecule such as siRNA, shRNA, miRNA and antisense RNA and dominant negative forms, designed to downregulate genes involved in or required for homologous recombination, for example, at least one of FANCA, NBS1, BRCA1, MSH2, RAD52, MRE11, DNA2, BRCA2, RAD51, TERF2, SRCAP, PALB2, SLX4, RAD54, RAD50, CtIP, TREX2, BRIP1, RANCD2, DCLRE1B, FANCE, FANCI, FANCL, EXO2, DMC1, RNF138, EXD2, KEAP1, XRCC2, XRCC3, RPA2, RPA1, PTEN, USP11, DSS1 and CHK1.

Each of the compositions described above can be encapsulated in nano-particles, for example lipid nano-particles.

Any of the RNA compositions of the present invention can be at least partially modified RNA, for example, RNA comprising 1-methyl pseudo-uridine or pseudo-uridine.

In some embodiments, RNAs of the present invention may be packaged in a virus for cellular delivery. Accordingly, a virus may be used to deliver a RNA composition to a cell. Any virus may be used for this purpose, including, but not limited to, DNA viruses, such as adeno-associated virus (AAV), and RNA viruses, such as lentivirus.

In an embodiment, the invention provides an exogenous RNA-based donor and its delivery to a target cell for genome editing. The exogenous RNA-based donor may be synthesized outside of a target cell by employing in-vitro transcription techniques or chemical synthesis. The exogenous RNA-based donor may also be produced in non-target cell and isolated for delivery to a target cell.

In some embodiments, the exogenous RNA-based donor is delivered as a naked RNA.

In an embodiment, the exogenous RNA-based donor is a non-coding ribonucleotide sequence.

In an embodiment, the exogenous RNA-based donor is devoid of a methylated cap at its 5′ termini. In an embodiment, the exogenous RNA-based donor comprises a non-methylated cap at its 5′ termini.

In an embodiment, the exogenous RNA-based donor is a non-naturally occurring RNA.

In one embodiment, the exogenous RNA-based donor is devoid of a 5′ UTR. In one embodiment, the RNA donor template is devoid of a 5′ ATG start codon of the open reading frame. In one embodiment, the exogenous RNA based donor is devoid of a 3′ poly-adenylated tail.

In some embodiments, the invention provides a composition of RNA molecules comprising:
a) a single stranded non-coding RNA comprising two homology arms, wherein the homology arms are designed to anneal or hybridize to genomic DNA sequences flanking an intended double-strand break site in a target DNA site; and
b) at least one of:
   (i) a mRNA encoding RNA-guided DNA nuclease (e.g., mRNA encoding Cas9);
   (ii) at least one guide RNA; and
   (iii) a tracr RNA.

In some embodiments, the composition of RNA molecules described above is introduced to a target cell as naked RNA molecules.

In an embodiment, the RNA-based donor is fused to or associated with a nucleotide motif capable of binding a functional polypeptide/protein. In an embodiment, the nucleotide motif is a RNA motif.

In one embodiment, the functional polypeptide/protein comprises a functional domain capable of modifying a target site of a genomic DNA sequence and a linking domain that binds to the RNA motif. In one embodiment, the functional polypeptide/protein is a nuclease (e.g., Cas9). In one embodiment, the functional polypeptide/protein is a fusion protein. In one embodiment, the fusion protein comprises a nuclease (e.g., Cas9, FokI, TALEN, and ZFN). Non-limiting examples of such proteins are described in PCT International Publication No. WO 2013/088446.

In a non-limiting example, a tracrRNA fused to or associated with the RNA-based donor of the invention may bind to a RNA-guided FokI Nuclease (RFN) fusion protein, wherein the RFN comprises a FokI catalytic domain sequence fused to the amino terminus of a catalytically inactive CRISPR-associated 9 protein (dCas9) such as disclosed in PCT International Publication No. WO 2014/144288.

In a further non-limiting example, the RNA motif is an MS2 binding site and the functional protein comprises a nuclease (e.g., Cas9) fused to an MS2 coat protein which recognizes and binds to the MS2 binding site, thereby facilitating association between the RNA based donor and the nuclease.

The present invention provides a composition comprising a RNA-based donor, wherein the RNA-based donor is a single stranded non-coding, non-translatable RNA correction template, wherein the RNA-based donor comprises homology arms designed to hybridize to target DNA sequences upstream and downstream of a intended double-strand break (DSB) site in a target DNA molecule. The homology arms are homologous to the sequences upstream and downstream to the DSB, however, the homology percentage may vary. The length of the homology arms may be 1-10, 10-50, 50-100, 100-250, 100-500, 100-1000 nucleotides or more. Furthermore, the length of the homology arm upstream of the DSB may differ from the length of the homology arm downstream of the DSB.

In one embodiment, the homology arm upstream of the intended DSB site in the target DNA comprises an insert sequence i.e., a region containing at least one difference in sequence relative to the target DNA sequence, which serves as a template for inducing sequence insertion(s), deletion(s) and/or substitution(s) in the target DNA.

In one embodiment, an insert sequence i.e., a region containing at least one difference in sequence relative to the target DNA sequence, overlaps the intended DSB site and is located between the homology arms.

In one embodiment, the homology arm downstream of the intended DSB site comprises an insert sequence.

In one embodiment, both the homology arm upstream of the intended DSB comprises an insert sequence and the homology arm downstream of the intended DSB comprises an insert sequence.

Accordingly, DNA repair mediated by a RNA templated repair mechanism which utilizes any one of the RNA-based donors described herein may result in: 1) an insertion of one or more continuous or discontinuous nucleotides to the genomic DNA, 2) a deletion of one or more continuous or discontinuous nucleotides the genomic DNA and/or 3) a substitution of one or more continuous or discontinuous nucleotides in the genomic target DNA.

The present invention provides a composition comprising a RNA template, comprising an insert sequence flanked by sequences having homology to an intended DNA target site.

The present invention provides a composition comprising a RNA template, comprising at least one insert sequence flanked by sequences having homology to a target DNA site sequence, wherein the at least one insert sequence contains at least one sequence difference relative to the target DNA site sequence, which at least one sequence difference is an alteration intended to be introduced into the target DNA site sequence.

In some embodiments, wherein the at least one sequence difference is:
a) a nucleotide or multiple nucleotides in the RNA template each of which is non-homologous or non-complementary to a corresponding nucleotide or multiple nucleotides of the target DNA site sequence;
b) a nucleotide or multiple nucleotides in the RNA template which do not have a corresponding nucleotide or multiple nucleotides in the target DNA site sequence;
c) an absence of a nucleotide or multiple nucleotides in the RNA template which correspond to a nucleotide or multiple nucleotides that are present in the target DNA site sequence; or
d) any combination of the above.

In some embodiments, wherein the RNA template is a non-naturally occurring RNA.

In some embodiments, wherein the RNA template comprises at least 10 nucleotides. The RNA template may be 10-12, 12-15, 15-18, 18-20, 20-25, 25-50, 50-100, 100-250, 250-500 or more basepairs in length.

In some embodiments, wherein the RNA template comprises a sequence having homology to a region upstream of a double-strand break in a DNA target site and a sequence having homology to a region downstream of said double-strand break in a DNA target site.

In some embodiments, wherein the at least one insert sequence is within a RNA template sequence having homology to a region upstream of a double-strand break in a DNA target site.

In some embodiments, wherein the at least one insert sequence is within a RNA template sequence having homology to a region downstream of a double-strand break in a DNA target site.

In some embodiments, wherein at least one insert sequence is within a RNA template sequence having homology to a region upstream of a double-strand break in a DNA target site and at least one insert sequence is within a RNA template sequence having homology to a region downstream of said double-strand break in a DNA target site.

In some embodiments, wherein at least one insert sequence overlaps a double-strand break in a DNA target site and is between a RNA template sequence having homology to a region upstream of the double-strand break and a RNA template sequence having homology to a region downstream of the double-strand break.

In some embodiments, wherein the RNA template comprises multiple insert sequences.

In some embodiments, wherein the RNA template is attached to at least one DNA molecule having sequence homology to the target DNA site.

In some embodiments, wherein the RNA template is attached to at least one self-annealing DNA molecule, which forms a hairpin loop.

In some embodiments, wherein the RNA template is attached to at least one DNA molecule, which contains a binding site for a transcription factor.

In some embodiments, wherein the transcription factor is capable of binding a region that regulates the expression of a gene containing the target DNA site.

In some embodiments, wherein the RNA template is attached to a DNA molecule, which contains a restriction enzyme binding site.

In some embodiments, wherein the RNA template is attached to a guide RNA capable of targeting a RNA-guided DNA nuclease.

In some embodiments, wherein a linker connects the RNA template to the guide RNA.

In some embodiments, wherein the RNA template is attached to a tracrRNA.

In some embodiments, wherein a linker connects the RNA template to the tracrRNA.

In some embodiments, wherein the RNA template is attached to a self-annealing RNA segment on at least one of its termini.

In some embodiments, wherein the RNA template is attached to a DNA molecule which encodes a recognition sequence that is specifically recognized by a DNA binding domain.

In some embodiments, wherein the attachment is a covalent linkage.

In some embodiments, wherein the attachment is by basepairing.

In some embodiments, wherein the RNA template contains a recognition sequence that is specifically recognized by a RNA binding domain.

In some embodiments, wherein the RNA template contains a cap.

In some embodiments, wherein the cap is a non-methylated cap.

In some embodiments, wherein the RNA template is unpolyadenylated.

In some embodiments, wherein the RNA template lacks a 5′ untranslated region.

In some embodiments, wherein the RNA template lacks a translation start site.

In some embodiments, wherein the target DNA is a eukaryotic genomic DNA.

In some embodiments, wherein the target DNA site is a transcribed region.

In some embodiments, wherein the target DNA site is an untranscribed region.

In some embodiments, wherein the target DNA site contains a PAM recognition sequence.

In some embodiments, wherein the RNA template is bound to a RNA binding protein to form a ribonucleoprotein.

In some embodiments, the composition further comprises at least one mRNA molecule.

In some embodiments, wherein the at least one mRNA molecule is connected to any one of the RNA templates described herein.

In some embodiments, wherein a cleavage sequence is present between the at least one mRNA molecule and the RNA template.

In some embodiments, wherein the mRNA molecule contains a cap.

In some embodiments, wherein the cap is a methylated cap.

In some embodiments, wherein the at least one mRNA molecule encodes a nuclease.

In some embodiments, wherein the nuclease is linked to an additional RNA-binding domain capable of specifically binding any one of the RNA templates described herein.

In some embodiments, wherein the nuclease is linked to an additional DNA-binding domain capable of specifically binding a DNA fragment attached to any one of the RNA templates described herein.

In some embodiments, wherein the nuclease is selected from the group consisting of a TALEN, a ZFN, a meganuclease and a RNA-guided DNA nuclease.

In some embodiments, the composition further comprises at least one nuclease.

In some embodiments, wherein the nuclease is linked to an additional RNA-binding domain capable of specifically binding any one of the RNA templates described herein.

In some embodiments, wherein the nuclease is linked to an additional DNA-binding domain capable of specifically binding a DNA fragment attached to a RNA template.

In some embodiments, wherein the nuclease is selected from the group consisting of a TALEN, a ZFN, a meganuclease and a RNA-guided DNA nuclease.

In some embodiments, the composition further comprises at least one guide-RNA capable of targeting a RNA-guided DNA nuclease.

In some embodiments, the composition further comprises at least one RNA interference molecule selected from the group consisting of a siRNA, a shRNA, a miRNA and an antisense RNA.

In some embodiments, wherein the at least one RNA interference molecule lowers expression of a gene involved in alternative end joining.

In some embodiments, wherein the at least one RNA interference molecule lowers expression of a gene involved in homologous recombination.

In some embodiments, wherein at least one of the RNA molecules of the composition is modified.

In some embodiments, wherein at least one of the RNA molecules contains at least one 1-methyl pseudo-uridine.

In some embodiments, wherein the composition is packaged for cellular delivery.

In some embodiments, wherein the package containing the composition is selected from the group consisting of virosomes, liposomes, immunoliposomes, polycation or lipid:nucleic acid conjugates, artificial virions, EnGeneIC delivery vehicles (EDVs), nano-particles and lipid nano-particles.

The present invention provides a pharmaceutical composition comprising any one of the compositions described herein.

The present invention also provides a host cell containing any one of the compositions described herein.

In some embodiments, wherein the genome of the cell has a double-strand break at the DNA target site which is targeted by the RNA template.

In some embodiments, wherein the cell is a post-mitotic cell.

In some embodiments, wherein the cell is selected from the group consisting of a myocyte, a cardiomyocyte, a hepatocyte, an osteocyte and a neuron.

In some embodiments, wherein the cell is a eukaryotic cell.

In some embodiments, wherein the cell is a mammalian cell.

In some embodiments, wherein the cell is a plant cell.

In some embodiments, wherein the cell is in culture.

In some embodiments, the present invention provides a host cell which has a genome edit relative to the genome of the host cell prior to delivery of any one of the compositions described herein, wherein the genome edit is encoded by the RNA template of said composition.

The present invention provides a method of genome editing in a cell comprising delivering to a cell any one of the compositions described herein. The present invention provides a method of gene editing a cell by providing an RNA template for correction of a target DNA sequence. Although a nuclease targeting said target DNA sequence may increase efficiency of the gene editing, a nuclease is not strictly required. Indeed, an RNA template alone may be provided to a cell for gene editing.

In some embodiments, wherein the delivery method is selected from the group consisting of electroporation, lipofection, microinjection, biolistics, particle gun acceleration, cationic-lipid mediated delivery and viral mediated delivery.

In some embodiments, wherein the cell is a post-mitotic cell.

In some embodiments, wherein the cell is a cell in a quiescent, non-dividing state.

In some embodiments, wherein the cell is selected from the group consisting of a myocyte, a cardiomyocyte, a hepatocyte, an osteocyte and a neuron.

In some embodiments, wherein the cell is a eukaryotic cell.

In some embodiments, wherein the cell is a mammalian cell.

In some embodiments, wherein the cell is a plant cell.

In some embodiments, wherein the delivery is selected from the group consisting of in vivo, in vitro and ex vivo delivery.

In some embodiments, wherein the cell is in culture.

In some embodiments, wherein the cell is in an organism.

In some embodiments, wherein the organism is a non-human organism.

In some embodiments, wherein the cell is genome edited by introducing an additional sequence to an intended target DNA site sequence within the cell.

In some embodiments, wherein the cell is genome edited by deleting a sequence from intended target DNA site sequence within the cell.

In some embodiments, wherein the cell is genome edited by substituting a sequence from intended target DNA site sequence within the cell.

The present invention provides a non-human transgenic organism formed by any one of the methods described herein.

The present invention also provides a kit comprising any one of the RNA templates described herein and instructions for use thereof.

In some embodiments, the kit further comprises any one of the compositions described herein.

In some embodiments, wherein the RNA template is in the same mixture as other molecules of the composition.

In some embodiments, wherein the RNA template is separated from other molecules of the composition.

The present invention provides the use of any one of the compositions described herein in the manufacture of a medicament.

The present invention also provides a method of treating a genetic disease in a patient comprising administering to the patient the pharmaceutical composition described above.

Any one of the compositions described herein can be comprised entirely of RNA molecules. Thus, an embodiment of the present invention is a composition comprising a RNA encoding nuclease e.g. a RNA-guided DNA nuclease such as Cas9, Cpf1, etc., a guide RNA used to target the nuclease and a donor RNA used as a template to repair the site targeted by the nuclease. Any one of these RNAs may be modified. Such modifications may, for example, lower or increase the RNA molecule's susceptibility to degradation. Alternatively, certain RNA modifications may influence the rate of translation of protein-encoding RNAs.

An embodiment of a composition useful for genome editing as described herein comprises two RNA molecules: (1) an mRNA encoding a nuclease and (2) a RNA-based donor. In one embodiment, the mRNA encoding a nuclease and the RNA-based donor may be directly ligated or otherwise connected, e.g., via basepairing, to each other, forming a single RNA molecule. As discussed in this specification, the RNA-based donor may be further connected to a guide/tracr RNA molecule to program a RNA-guided DNA nuclease, e.g., Cas9, Cpf1, etc.

Furthermore, in one embodiment a cleavage sequence may be present between the mRNA portion and RNA-based donor portion of such a single RNA molecule, such that the two portions are separable upon appropriate conditions for cleavage e.g., in a cell. Several cleavage sequences are known in the art, such as self-cleaving ribozyme sequences, hammerhead ribozyme sequences, hairpin ribozyme sequences, etc.

Each embodiment disclosed herein is contemplated as being applicable to each of the other disclosed embodiments. Thus, all combinations of the various elements described herein are within the scope of the invention.

### BRIEF DESCRIPTION OF THE FIGURES

FIG. 1A-FIG. 1C: Examples of RNA-based donors.

FIG. 1A shows one basic design of a RNA-based donor. In this case an insert sequence is flanked on both sides by homology arms, which each share sequence homology with DNA target region.

FIG. 1B further shows the addition of DNA sequences on either end of the RNA-based donor. Such DNA sequences also share sequence homology to the DNA target region.

FIG. 1C shows the addition of a self-annealing RNA hairpin at the 5′ terminus of the RNA-based donor. Such RNA self-annealing hairpins may be placed at either end or both ends of the RNA donor.

FIG. 2A-FIG. 2D: Schematic description of RNA-based donors linked to transcription factors (TF) or restriction enzyme binding sites.

FIG. 2A shows an embodiment of a RNA-based donor wherein the donor region is flanked on the 5′ end by a self-annealing DNA hairpin and flanked on the 3′ end by a self-annealing DNA hairpin which contains a transcription factor binding site.

FIG. 2B shows an embodiment of a RNA-based donor wherein the donor region is capped by an ApppG/GpppG at the 5′ end and flanked on the 3′ end by a self-annealing DNA hairpin containing a transcription factor binding site.

FIG. 2C shows an embodiment of a RNA-based donor wherein the donor region is capped by an ApppG/GpppG at the 5′ end and flanked on the 3′ end by a self-annealing DNA hairpin containing a restriction enzyme recognition site.

FIG. 2D shows an embodiment of a RNA-based donor wherein the donor region is capped by an ApppG/GpppG at the 5′ end and flanked on the 3′ end by a RNA/DNA hybrid hairpin containing a restriction enzyme recognition site.

FIG. 3A-FIG. 3E: Embodiments of RNA-based single molecule donor RNA, guide RNA and tracrRNA.

FIG. 3A shows an embodiment of a RNA-based donor which is directly ligated to a downstream poly-(CAA)ₙ linker, which is further directly ligated to a downstream single-guide RNA capable of activating a RNA-guided DNA nuclease, e.g., Cas9, Cpf1 etc. In the shown embodiment, the RNA-based donor is flanked on its 5′ end by a DNA having a sequence homologous to the corresponding target sequence.

FIG. 3B shows an embodiment similar to FIG. 3A, however the RNA-based donor is flanked on its 5′ end by a self-annealing DNA hairpin.

FIG. 3C also shows an embodiment similar to FIG. 3A, however the RNA-based donor is flanked on its 5′ end by a self-annealing RNA hairpin.

FIG. 3D also shows an embodiment similar to FIG. 3A, however the RNA-based donor is capped by an ApppG/GpppG at the 5′ end (as symbolized by a filled circle).

FIG. 3E shows an embodiment wherein a tracr RNA is ligated at its 3′ end to a poly-(CAA)ₙ linker, which is further directly ligated to a downstream RNA-based donor. In this embodiment, the guide-RNA is capped by an ApppG/GpppG at the 5′ end (as symbolized by a filled circle). Although FIG. 3A-FIG. 3E depicts the RNA-based donor, linker, guide-RNA and tracrRNA as directly ligated to each other, these portions may be attached to each other via overlapping, complementary basepairs.

FIG. 4A and FIG. 4B: Experimental design to measure transcription-linked error-free NHEJ.

FIG. 4A shows a schematic diagram of an assay to measure genome edits made from a RNA correction template. A dsDNA construct encoding a GFP donor template 88, 144 or 312 nucleotides in length is placed under the transcriptional control of a U6 polIII promoter and serves as a “GFP RNA-donor” construct. The “GFP RNA-donor” construct, a construct expressing Cas9 and a construct expressing a guide RNA targeting the Cas9 to the inactive GFP target site are each transfected into Hek293 cells stably expressing inactive GFP. Error-free NHEJ events utilizing the “GFP RNA-donor”as a template are measured by GFP positive cells. To ensure the GFP positive cells are derived from transcription-linked error-free NHEJ events, a control construct is created by removing the U6 promoter from the “GFP RNA-donor” construct via KpnI digestion, thereby eliminating transcription of the GFP donor template, and is transfected into Hek293 cells stably expressing inactive GFP. Accordingly, GFP positive cells that are transfected with the control construct are derived from HDR utilizing the dsDNA construct itself as a template. The number of GFP positive cells transfected with the control construct are used to normalize results from the “GFP RNA-donor” construct.

FIG. 4B shows a portion of the target dsDNA construct sequence described above, which encodes an inactive GFP (iGFP). Stop codons are indicated by (*). A guide RNA capable of targeting the iGFP is also shown. After a Cas9-induced double-strand break is formed at the target site, a “GFP RNA-donor” is used as a RNA correction template during DNA repair to remove the premature stop codons and form a sequence encoding full-length GFP.

FIG. 5: Transcription-linked error-free NHEJ in HEK293 cells.

Utilizing the method shown in FIG. 4A, varying amounts (50, 100 or 200 ng) of the GFP RNA-donor construct or control construct were transfected into Hek293 cells stably expressing inactive GFP. The number of GFP positive cells were normalized by transfection efficiency.

FIG. 6A and FIG. 6B: Experimental design to test the effect of donor transcript proximity to the DSB site on the efficiency error-free NHEJ repair.

FIG. 6A—The GFP assay described in FIG. 4A was used to determine the effect of bringing the donor transcript into close proximity of a DSB site. In order to bring the RNA-based donor into close proximity of the target site, a dsDNA construct encoding a GFP donor template directly linked to a downstream poly-(CAA)ₙ linker, which is further directly linked to a downstream guide RNA capable of targeting Cas9 to the inactive GFP target site, which is further linked to a downstream tracrRNA, was generated. The donor encoding region is placed under the transcriptional control of a U6 polIII promoter. The construct is referred to as the “fused 5′ GFP donor+gRNA construct.” The fused 5′ GFP donor+gRNA construct and a construct expressing the Cas9 are each transfected into Hek293 cells stably expressing inactive GFP. Briefly, error-free NHEJ events utilizing the “fused 5′ GFP donor+gRNA” region as a template is measured by GFP positive cells. To ensure the GFP positive cells are derived from transcription-linked error-free NHEJ events, a control construct is created by removing the U6 promoter from a GFP donor construct via KpnI digestion, thereby eliminating transcription of the GFP donor template. Accordingly, GFP positive cells that are transfected with the control donor construct are derived from HDR utilizing the dsDNA construct itself as a template. The percentage of GFP positive cells was normalized to cells containing a plasmid expressing CFP in order to determine transfection efficiency. To test the effect of bringing the donor transcript in close vicinity to the DSB site, the results are further compared to error-free NHEJ events utilizing the “GFP RNA-donor” as a template described in FIG. 4A and FIG. 4B.

FIG. 6B—An additional dsDNA construct encoding a GFP donor template directly linked to an upstream poly-(CAA)ₙ linker, which is further linked to an upstream tracrRNA, which is further directly linked to an upstream guide RNA capable of targeting Cas9 to the inactive GFP target site, was generated. The donor encoding region is placed under the transcriptional control of a U6 polIII promoter. The construct is referred to as the “fused gRNA+GFP donor 3′” construct. The “fused gRNA+GFP donor 3′” construct and a construct expressing the Cas9 are each transfected into Hek293 cells stably expressing inactive GFP. Briefly, error-free NHEJ events utilizing the “fused gRNA+GFP donor 3′” region as a template is measured by GFP positive cells. To ensure the GFP positive cells are derived from transcription-linked error-free NHEJ events, a control construct is created by removing the U6 promoter from a GFP donor construct via KpnI digestion, thereby eliminating transcription of the GFP donor template. Accordingly, GFP positive cells that are transfected with the control donor construct are derived from HDR utilizing the dsDNA construct itself as a template. The percentage of GFP positive cells was normalized to cells containing a plasmid expressing CFP in order to determine transfection efficiency. To test the effect of bringing the donor transcript in close vicinity to the DSB site, the results are further compared to error-free NHEJ events utilizing the “GFP RNA-donor” as a template as described in FIG. 4A and FIG. 4B.

FIG. 7: Data demonstrating effect of donor transcript proximity to the DSB site on the efficiency error-free NHEJ repair in HEK293 cells.

Utilizing the method shown in FIG. 4A and FIG. 6A, the “fused 5′ GFP donor+gRNA” construct, the “GFP RNA-donor” construct or the “GFP RNA-donor” control construct lacking a U6 promoter were transfected with or without Cas9 into Hek293 cells stably expressing inactive GFP. The number of GFP positive cells were normalized by transfection efficiency.

FIG. 8A-FIG. 8C: Gene editing via the transcription-linked error-free NHEJ pathway.

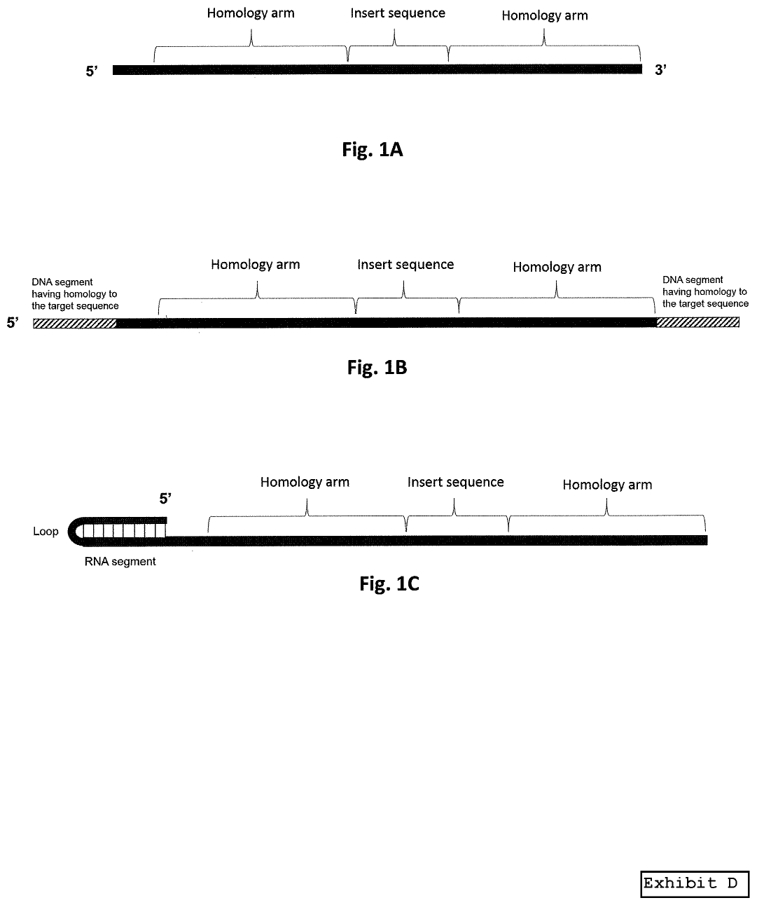

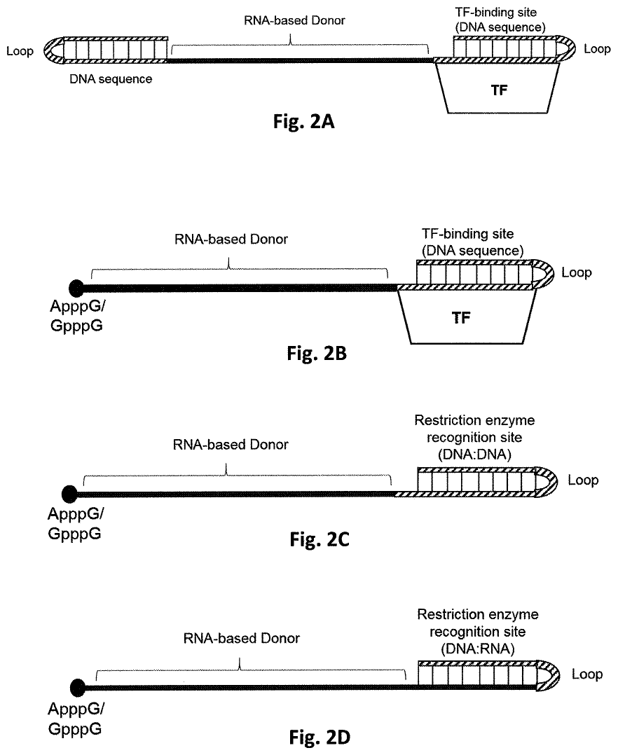

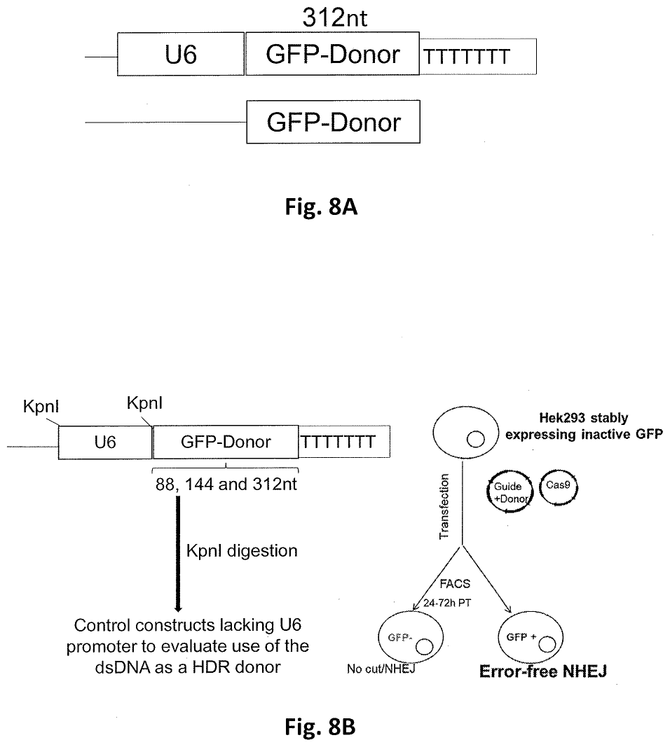

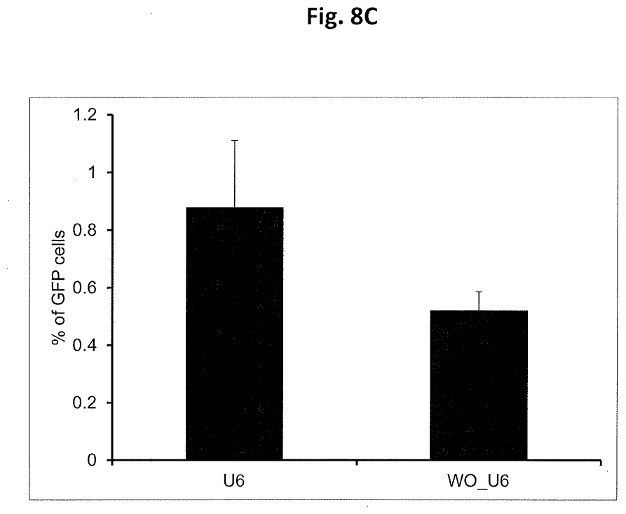

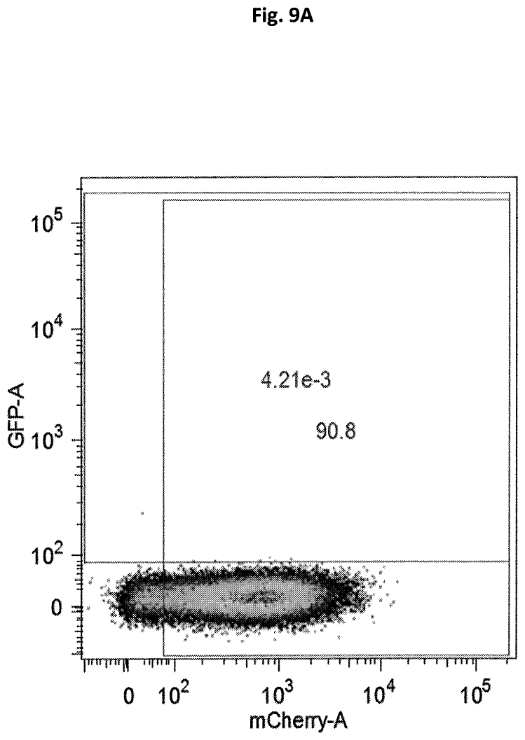

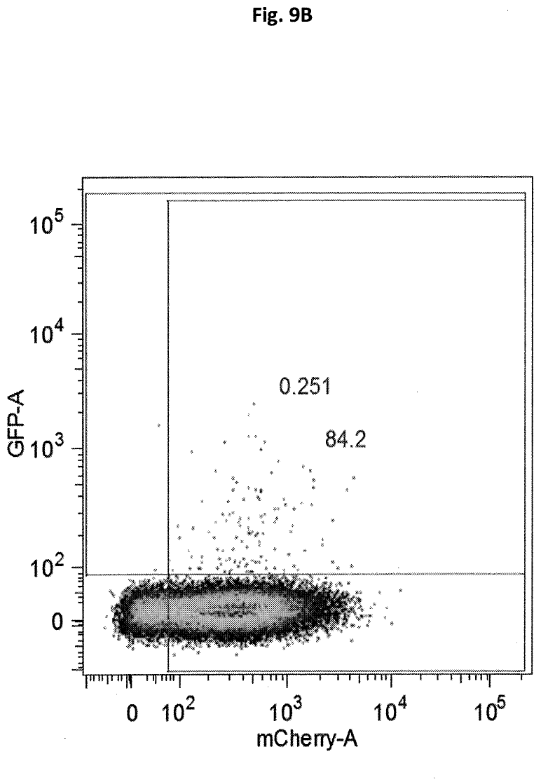

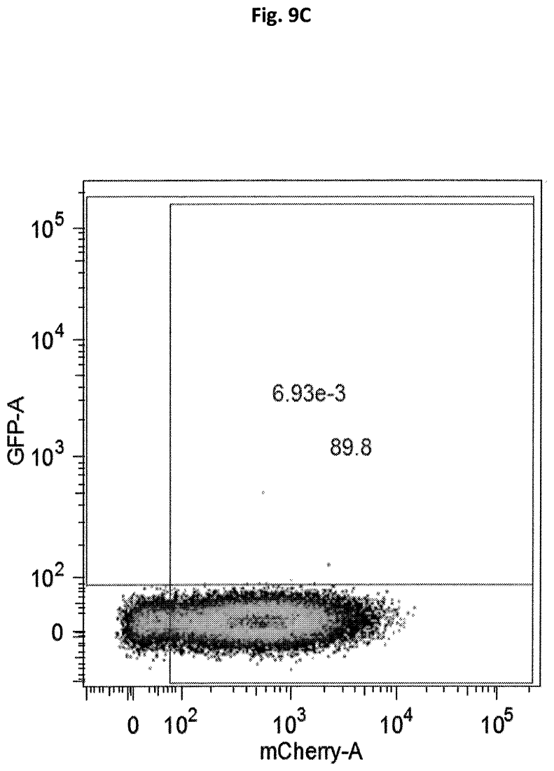

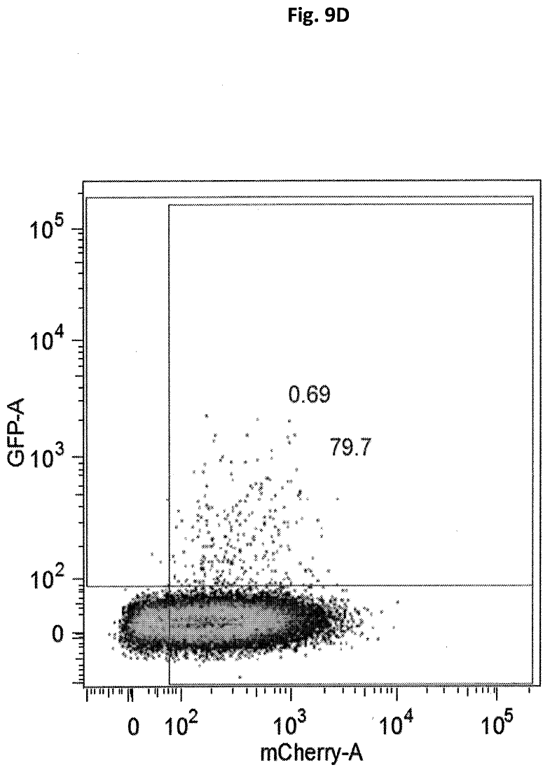

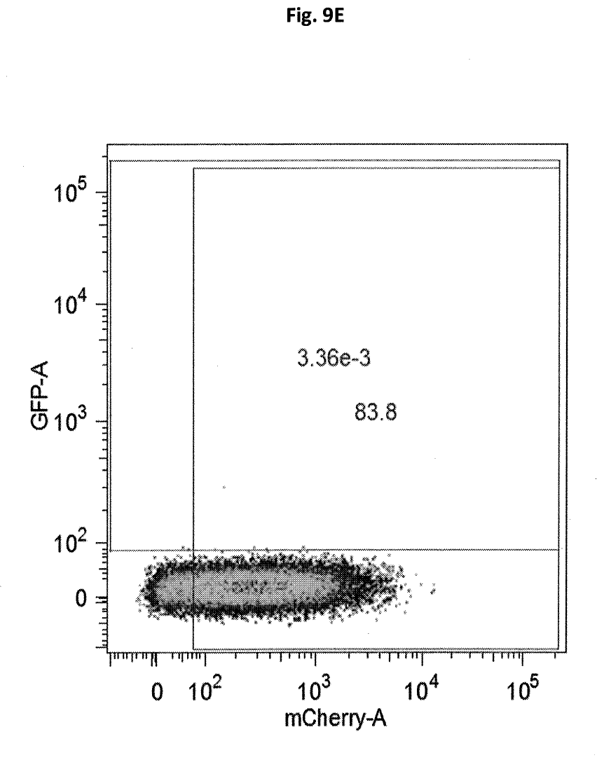

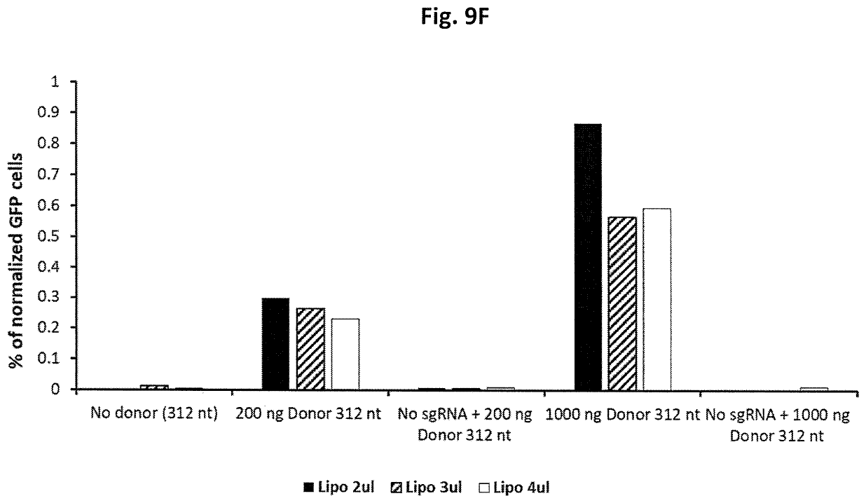

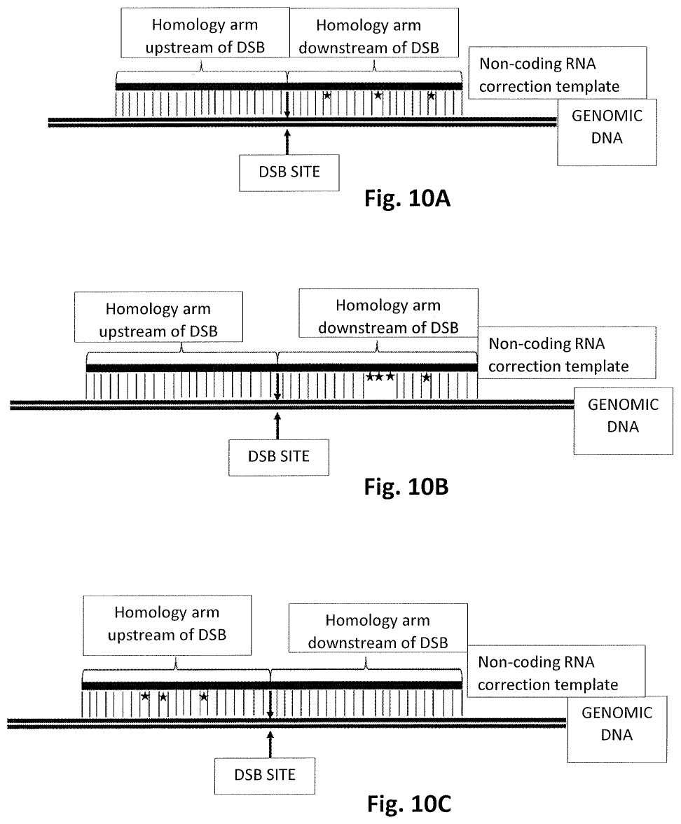

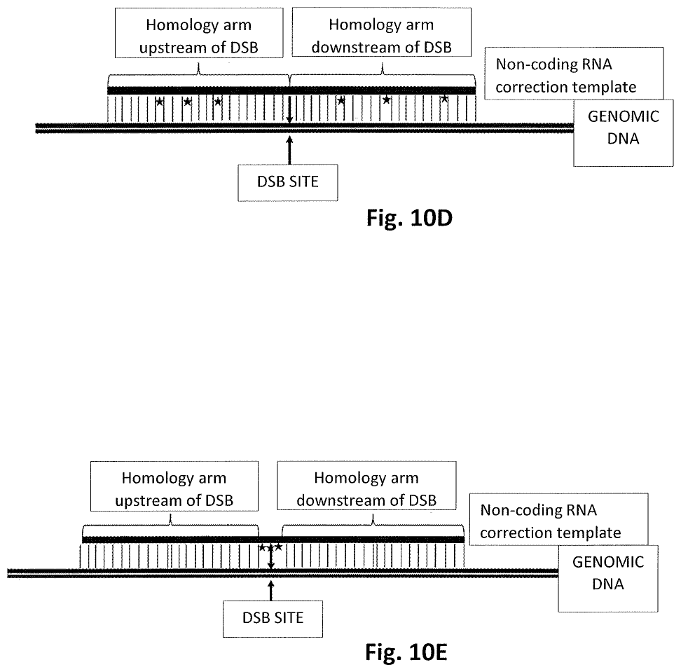

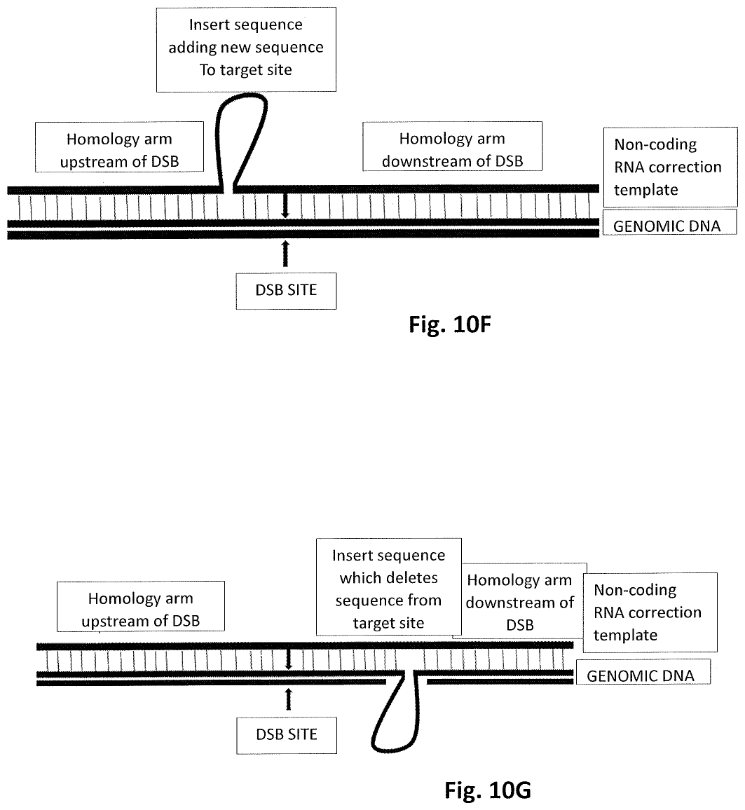

As a proof of concept for utilizing the transcription-linked error-free NHEJ pathway for gene editing, we prepared a construct expressing a GFP-donor sequence comprising the N-terminus sequence of EGFP under the control of a U6 promoter. As a control, the U6 promoter was excluded (FIG. 8A). The constructs were co-transfected with a plasmid expressing Cas9 and gRNA into HEK-293 cells expressing inactive GFP (FIG. 8B). 72 h post transfection the cells were harvested and the percentage of GFP positive cells was measured by FACS. Cells that were transfected with the U6-GFP-Donor indicate the efficiency of error-free NHEJ. The control cells (transfected with the control construct i.e., without the U6 promoter) indicate the HDR rates. The graph summarizes the mean±S.D of 4 independent experiments. * P<0.05 as determined by T-test (FIG. 8C).

FIG. 9A-FIG. 9F: Induction of Cas9-mediated error-free NHEJ using RNA components.

FIG. 9A—Hek293 cells stably expressing inactive GFP (iGFP-Hek293) were transfected with RNA components using 2 μl Lipofectamine 3000. Control cells were transfected with 500 ng mRNA encoding Cas9, 100 ng sgRNA targeting the inactive GFP sequence and 500 ng mRNA encoding mCherry only. No donor template was provided.

FIG. 9B—In an experimental sample, iGFP-Hek293 cells were transfected with 500 ng mRNA encoding Cas9, 100 ng sgRNA targeting the inactive GFP sequence, 500 ng mRNA encoding mCherry and 200 ng of 312 nt GFP RNA donor.

FIG. 9C—As a control, iGFP-Hek293 cells were transfected with 500 ng mRNA encoding Cas9, 500 ng mRNA encoding mCherry and 200 ng of 312 nt GFP donor. No sgRNA was provided.

FIG. 9D—In another experimental sample, iGFP-Hek293 were transfected with 500 ng mRNA encoding Cas9, 100 ng sgRNA targeting the inactive GFP sequence, 500 ng mRNA encoding mCherry and 1000 ng of 312 nt GFP RNA donor.

FIG. 9E—As a control, iGFP-Hek293 were transfected with 500 ng mRNA encoding Cas9, 500 ng mRNA encoding mCherry and 1000 ng of 312 nt GFP donor. No sgRNA was provided.

FIG. 9F—A graph quantifying RNA-templated repair in cells transfected with the RNA components listed in FIG. 9A-FIG. 9E using varying amounts of Lipofectamine 3000.

FIG. 10A-FIG. 10G: Examples of inserts within a RNA-based donor.

FIG. 10A shows one example of an insert sequence within a RNA-based donor. The RNA-based donor serves as a non-coding RNA correction template that hybridizes to a genomic DNA target region upstream and downstream of an intended DSB site. The RNA-based donor template shares sequence homology with a genomic DNA target region yet also contains differences in sequence relative to the genomic DNA target region. Such sequence differences, represented by stars in FIG. 10A-FIG. 10E, are considered inserts, or part of an insertion sequence, and are introduced into the genomic DNA target region during RNA-templated repair.

FIG. 10B shows a RNA-based donor wherein the sequence differences of the insertion sequence are not evenly distributed throughout the downstream homology arm.

FIG. 10C shows a RNA-based donor wherein the insertion sequence is located in the upstream homology arm.

FIG. 10D shows a RNA-based donor wherein the insertion sequences are located in both the upstream and downstream homology arms.

FIG. 10E shows a RNA-based donor, wherein the insertion sequence overlaps the DSB site and is between the upstream and downstream homology arms.

FIG. 10F shows a RNA-based donor, wherein the insertion sequence of the RNA template is a new sequence that was not originally present in the DNA target sequence.

FIG. 10G shows a RNA-based donor, wherein the RNA template removes a sequence that was originally present in the DNA target sequence. The RNA template lacks the corresponding sequence of the DNA target sequence.

### DETAILED DESCRIPTION OF THE INVENTION

Described herein are compositions and methods for increasing the effectiveness of gene editing by delivering RNA compositions, including RNA-based donors, to cells.

### Terms

Unless otherwise defined, all technical and scientific terms used herein have the same meaning as commonly understood by a person of ordinary skill in the art to which this invention belongs.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acid.

The term “targeted insertion” as used herein refers to the result of a successful DNA repair event wherein a desired portion of a donor RNA sequence was inserted or copied into a desired position in the genome of a cell. Thus, the terms “insert,” “insertion sequence” and “insert sequence” refer to a sequence of a donor template which is desired to be inserted, copied, incorporated or otherwise introduced into a desired position in the genome of a cell. An insert sequence may be any length and preferably differs from the original DNA target site sequence by at least one basepair. For instance, a RNA-based donor may serve as a RNA correction template containing at least one insertion sequence, wherein the at least one insertion sequence contains at least one difference in sequence relative to the sequence of the DNA target site, resulting in an alteration to the original DNA target site sequence.

The term “sequence difference” or “difference in sequence” as used herein refers any portion of a RNA donor sequence that differs from the DNA target sequence. Such sequence differences belong to the insertion sequence of the RNA-based donor. For example, a sequence difference may be a nucleotide of the RNA-based donor that does not form a natural basepair i.e., A-T(U), T(U)-A, C-G or G-C, with the corresponding DNA target nucleotide. Such a sequence difference in the RNA-based donor results in a nucleotide substitution of the original target DNA sequence.

The sequence RNA-based donor may differ in the number of nucleotides relative to the sequence of the target DNA sequence, resulting in an addition of new sequence to the DNA target sequence or deletion of a portion of the original DNA target sequence.

For instance, a RNA-based donor sequence may lack corresponding portions of the DNA target sequence entirely and is thus shorter in length than the corresponding DNA target sequence. Such a sequence difference would be represented in a sequence alignment of the RNA-based donor and the target DNA site as a gap in the RNA-based donor. Accordingly, use of such a RNA-based donor for genome editing would result in a deletion of the missing sequence from the original target DNA sequence.

Conversely, a RNA-based donor sequence may contain additional nucleotides relative to the corresponding target DNA sequence and is thus longer than the corresponding target DNA sequence. Such a sequence difference would be represented in a sequence alignment of the RNA-based donor and the target DNA as a gap within the target DNA sequence. Accordingly, use of such a RNA-based donor for genome editing would result in the introduction of the additional sequence into the original target DNA sequence.

The term “off-target excision of the genome” as used herein refers to the percentage of cells in a cell population where the DNA of a cell was excised by a nuclease at an undesired locus during or as a result of genome editing. The detection and quantification of off-target insertion events can be done by known methods.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” or “TALEN” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. As a non-limiting example see, for example, U.S. Pat. No. 8,586,526.

Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a non-naturally occurring protein whose design/composition results principally from rational criteria. Rational criteria for design include application of substitution rules and computerized algorithms for processing information in a database storing information of existing ZFP and/or TALE designs and binding data. See, for example, U.S. Pat. Nos. 8,586,526; 6,140,081; 6,453,242; and 6,534,261; see also WO 98/53058; WO 98/53059; WO 98/53060; WO 02/016536 and WO 03/016496.

A “selected” zinc finger protein or TALE is a protein not found in nature whose production results primarily from an empirical process such as phage display, interaction trap or hybrid selection. See e.g., U.S. Pat. Nos. 8,586,526; 5,789,538; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084.

“DNA break” refers to both a single strand break (SSB) and a double strand break (DSB). A SSB is a break that occurs in one DNA strand of a double helix and can be caused by, for instance, nickase activity. A DSB is a break in which both DNA strands of a double helix are severed.

“DNA cleavage” refers to the breakage of the covalent backbone of a DNA molecule. DNA cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events at two adjacent loci in the genome. DNA cleavage can result in the production of either blunt ends or staggered ends. In the present invention, DNA cleavage may be targeted to a region of interest in order to induce cellular pathways which introduce a sequence from an exogenous RNA-based donor into a target site.

The term “nucleotide sequence” refers to a nucleotide sequence of any length, which can be DNA or RNA, can be linear, circular or branched and can be either single-stranded or double-stranded. The term “sequence” refers to the sequence information encoded by a nucleotide molecule. Accordingly, a sequence from a RNA-based donor can be inserted, copied, incorporated or introduced into a target DNA sequence by any mechanism.

The term “donor sequence” or “donor template” refers to a nucleotide sequence that is inserted or copied into a genome. Notably, the donor molecule itself may not be inserted, but rather used as a template such that the sequence it encodes may be copied into a target site. A donor sequence can be of any length, for example between 2 and 10,000 nucleotides in length (or any integer value there between or there above), preferably between about 100 and 1,000 nucleotides in length (or any integer there between), more preferably between about 200 and 500 nucleotides in length.

The term “homology portion of the donor” as used herein refers to a sequence of the RNA-based donor which is partially or fully homologous, i.e. sharing sequence homology, to the target site in the genome.

The term “RNA-based donor” refers to a donor template that is comprised of RNA. Specifically, the sequence which is inserted or copied into the genome during repair is derived from a RNA template. However, the RNA-based donor molecule may be attached to other types of nucleotides e.g., DNA.

The RNA-based donor comprises at least one insertion sequence flanked by homology portions of any length. The RNA-based donor insertion sequence(s) are considered as any sequence which differs from the target DNA sequence. The RNA-based donor serves as a template to edit the target DNA sequence, and thus may also be referred to as a RNA correction template. The insertion sequence of the RNA-based donor may be designed to add, delete or substitute bases in the original DNA target sequence.

An “exogenous” molecule is a molecule that is not normally present in a cell, but can be introduced into a cell by one or more genetic, biochemical or other methods.

“Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Plant” cells include, but are not limited to, cells of monocotyledonous (monocots) or dicotyledonous (dicots) plants. Non-limiting examples of monocots include cereal plants such as maize, rice, barley, oats, wheat, sorghum, rye, sugarcane, pineapple, onion, banana, and coconut. Non-limiting examples of dicots include tobacco, tomato, sunflower, cotton, sugarbeet, potato, lettuce, melon, soybean, canola (rapeseed), and alfalfa. Plant cells may be from any part of the plant.

“Eukaryotic” cells include, but are not limited to, fungal cells (such as yeast), plant cells, animal cells, mammalian cells and human cells.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example an engineered nuclease. Engineered nuclease technology is based on the engineering of naturally occurring DNA-binding proteins, including ZFPs and TALEs.

The term “homology directed repair” (HDR) refers to a mechanism by which cells repair DNA damage (double strand DNA lesions and single strand nicks). The most common form of HDR is homologous recombination (HR). Although HDR has typically been described as using DNA e.g., a sister chromatid, as a template, RNA-templated repair has also been described. See Storici et al., 2007, Nature. Vol. 447, pgs. 338-341, hereby incorporated by reference. Furthermore, transcript-RNA-templated DNA repair has been described in Keskin et al., 2014, Nature, Vol. 515, pgs. 436-439, hereby incorporated by reference.

The term “non-homologous end joining” (NHEJ) refers to another cellular mechanism to repair DNA damage. There are two distinct NHEJ pathways, alternative end joining and classical NHEJ, each of which utilize distinct sets of proteins. While alternative end joining is an error prone process, RNA may be used as a template during the classical NHEJ pathway. See Chakraborty et al., 2016, Nature Commun., 7:13049, hereby incorporated by reference. Thus, in one aspect of the invention methods to bias cellular DNA repair towards classical NHEJ are envisioned, including adding promoting factors of classical NHEJ and/or inhibiting factors e.g., siRNA, of HR and/or alternative NHEJ pathways.

Any cellular mechanism of DNA-damage repair which utilizes a RNA-based donor template as described herein, directly or indirectly e.g., via a cDNA intermediate, are contemplated as being utilized for the genome editing methods described throughout this application.

In certain embodiments, a nuclease which may be used to generate DNA breaks and initiate cellular DNA repair pathways comprises a ZFN, TALEN or meganuclease.

In certain embodiments, a nuclease which may be used to generate DNA breaks and initiate cellular DNA repair pathways comprises a CRISPR/Cas system. The CRISPR (clustered regularly interspaced short palindromic repeats) locus, which encodes RNA components of the system, and the cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

The term guide RNA (gRNA) refers to a RNA molecule capable of forming a complex with a CAS protein e.g., Cas9 and wherein said complex is capable of targeting a DNA sequence i.e., genomic DNA sequence having a nucleotide sequence which is complementary to said gRNA. In some embodiments, gRNA is an approximately 20 bp RNA molecule that can form a complex with Cas9 and serve as the DNA recognition module. A guide RNA can be custom designed to target any desired sequence.

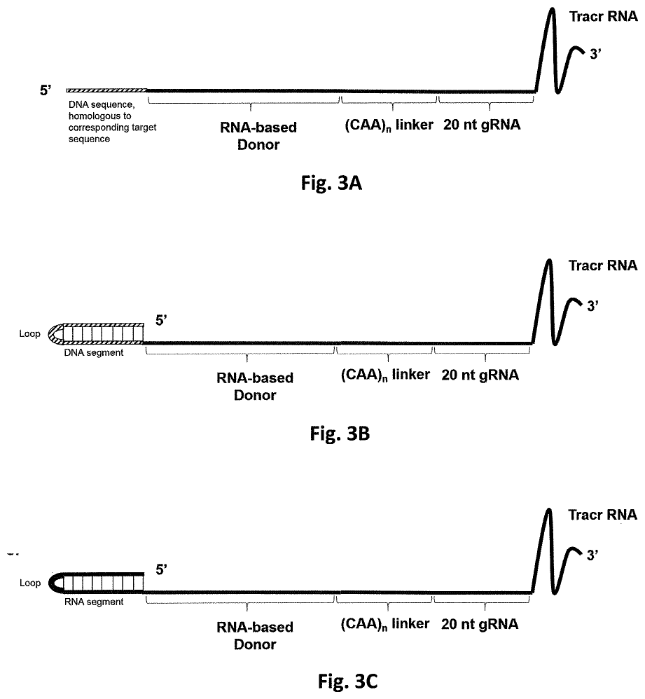

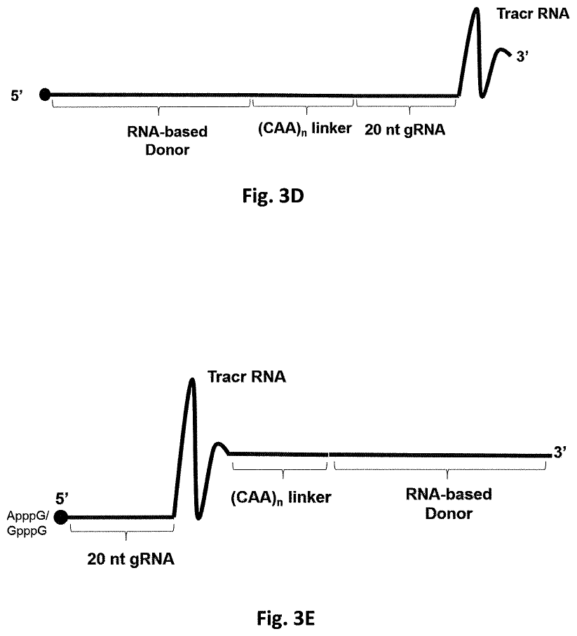

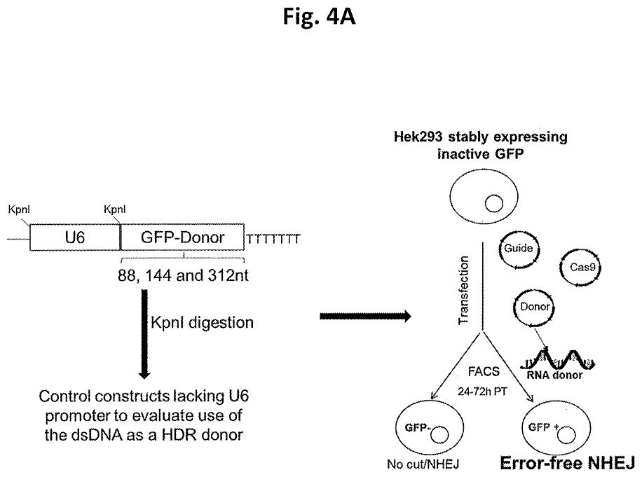

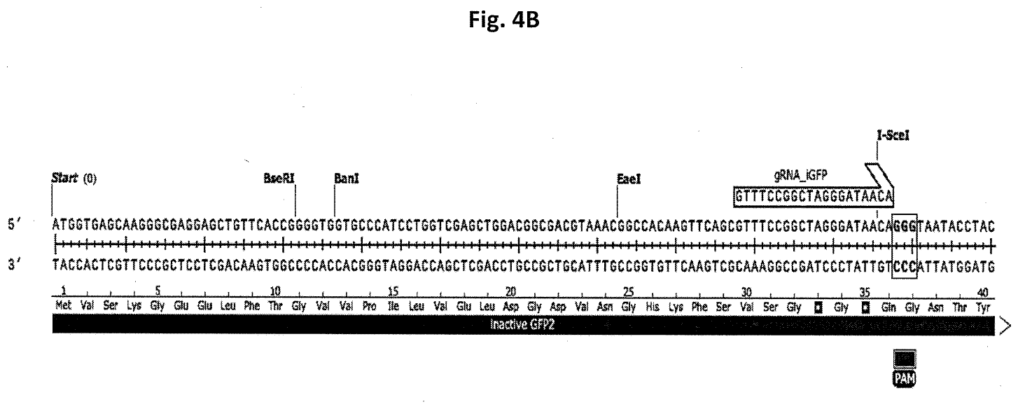

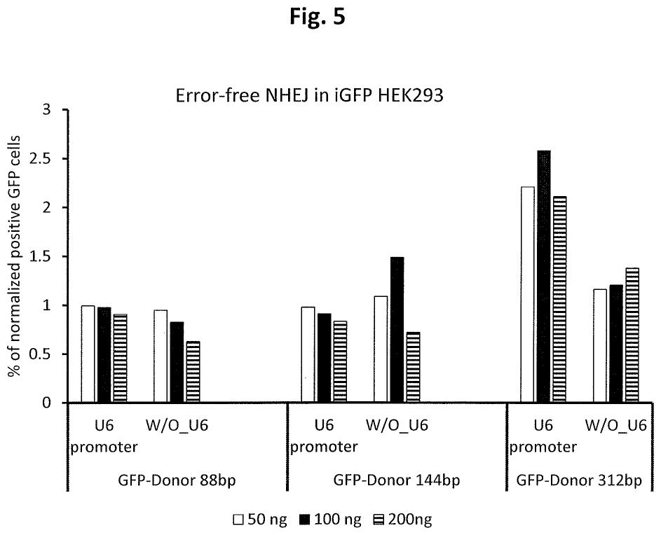

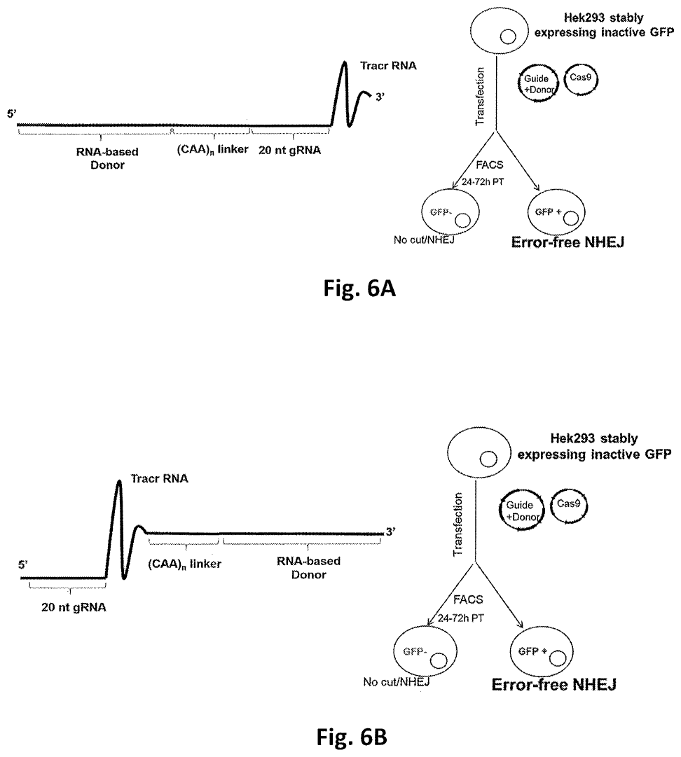

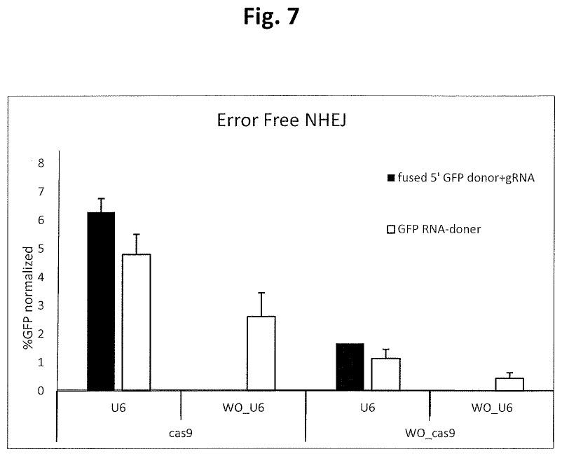

The term “single guide RNA” (sgRNA), is a RNA molecule that can form a complex with Cas9 and serve as the DNA recognition module. sgRNA is designed as a synthetic fusion of the CRISPR RNA (crRNA, or guide RNA) and the trans-activating crRNA (tracrRNA). A sgRNA may be connected to a RNA-based donor sequence on the same molecule. Accordingly, the RNA-based donor will be in close proximity to the target site bound and cut by the RNA-guided DNA nuclease activated and targeted by the sgRNA. A linker may separate the RNA-based donor sequence from the sgRNA. See FIG. 3A-FIG. 3E, for example. A sgRNA is not strictly required, as the use of separate guide RNA and tracrRNA molecules which connect to each other via basepairing is also considered and may be advantageous in certain applications of the invention described herein.

Increasing the concentration of donor nucleic acid near the target site of a nuclease may also be achieved by physically linking the donor nucleic acid to the nuclease. In such an embodiment, the nuclease may be fused to an additional domain which specifically binds the donor nucleic acid. See PCT International Application Nos. WO/2016/0653464 and WO/2016/054326. In certain embodiments, a DNA construct which encodes a RNA donor template for RNA-templated DNA repair is physically linked to the nuclease. Accordingly, transcription of the RNA donor template off of the DNA construct increases the local concentration of said RNA donor template at the nuclease target site.

Any DNA binding domain (DBD) may be fused to a nuclease in order to specifically bind a DNA encoding a RNA-based donor template, or a DNA fragment connected to a RNA-based donor template. Thus, “DNA binding domain” refers to any peptide or polypeptide that has the ability to bind DNA in a sequence specific manner. Different types of DNA binding domains are known in the art. In some embodiments, the DBD of the present invention may be selected from the group consisting of: Helix-turn-helix, zinc finger, leucine zipper, winged helix, helix-loop-helix, HMG box, Wor3 domain, OB-fold domain and B3 domain, among others. In some embodiments, the DNA binding domain which binds a DNA encoding a RNA-based donor template, or a DNA fragment connected to a RNA-based donor template, may be any DBD known in the art. In some embodiments, the at least one additional DNA binding domain may be a catalytically inactive RNA-guided DNA nuclease which binds a DNA encoding a RNA-based donor template, or DNA fragment connected to a RNA-based donor, via an appropriate guide-RNA. Thus, in some embodiments a RNA-based donor is attached to a DNA molecule which encodes a recognition sequence that is specifically recognized by a DNA binding domain. Similarly, a DNA encoding the RNA-based donor may also encode a recognition sequence that is specifically recognized by a DNA binding domain.

Furthermore, there are numerous databases listing DNA binding domains as well various software for predicting the capacity of DNA binding of a peptide based on its sequence. For example UniProt database includes information of the DNA binding properties of proteins and peptides. The DNA binding domain includes any peptide which is either known as a DNA binding peptide or is predicted to be a DNA binding peptide by its sequence.

Non-limiting examples for DBDs are described in WO01/92501, U.S. Publication No. 2004/0219558, PCT/US2012/065634, PCT/US1995/016982, U.S. Pat. Nos. 9,017,967 and 7,666,591 all of which are herein incorporated in their entirety.

In other embodiments, a RNA-based donor template or multiple copies thereof are linked to the nuclease via a RNA binding domain that is fused to a nuclease and which specifically binds the RNA-based donor template. Different types of RNA binding domains are known in the art. In some embodiments, the RNA binding domain linked to the nuclease may be selected from the group consisting of bacteriophage Phi21 Nprotein, PUF5 binding element, viral TAT proteins, MS2 coat protein and Cys4, among others. The RNA binding domain includes any peptide which is either known as a RNA binding peptide or is predicted to be a RNA binding peptide by its sequence. In some embodiments, the at least one additional RNA binding domain may be a catalytically inactive RNA-guided DNA nuclease which binds the RNA-based donor via a guide-RNA that is directly linked the RNA-based donor. Thus, in some embodiments a RNA-based donor encodes a recognition sequence that is specifically recognized by a RNA binding domain.

### Overview

### Nucleases

Any nuclease may be used to create DNA damage and consequently initiate cellular DNA repair. An mRNA encoding a nuclease may be delivered to a cell, such that the nuclease is capable of being expressed in the cell. However, a nuclease may be directly delivered to a cell or, alternatively, a DNA encoding a nuclease may be delivered to a cell such that the nuclease is capable of being expressed in the cell.

A nuclease domain may be linked to a DNA binding domain which specifically binds a DNA target site of interest. Thus, a DNA binding domain may confer specificity to a nuclease domain. Several non-limiting examples of DNA-binding domains which may be used for this purpose are described below.

In certain embodiments, a DNA binding domain comprises a zinc finger protein. Preferably, the zinc finger protein is non-naturally occurring in that it is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061.

In certain embodiments, the DNA binding domain is an engineered zinc finger protein that typically includes at least one zinc finger but can include a plurality of zinc fingers (e.g., 2, 3, 4, 5, 6 or more fingers). Usually, the ZFPs include at least three fingers. Certain of the ZFPs include four, five or six fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more regulatory domains, wherein these regulatory domains can be transcriptional activation or repression domains.

In other embodiments, the DNA binding domain comprises a TALE DNA binding domain (as a non-limiting example see, U.S. Pat. No. 8,586,526). The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay et al. (2007) Science 318:648-651). These proteins contain a DNA binding domain and a transcriptional activation domain. One of the most well characterized TALEs is AvrBs3 from Xanthomonas campestgris pv. Vesicatoria (see Bonas et al (1989) Mol Gen Genet. 218: 127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al. (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al. (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Thus, in some embodiments, the DNA binding domain that binds to a target site in a target locus (e.g., globin or safe harbor) is an engineered domain from a TAL effector similar to those derived from the plant pathogens Xanthomonas (see Boch et al., (2009) Science 326: 1509-1512 and Moscou and Bogdanove, (2009) Science 326: 1501) and Ralstonia (see Heuer et al (2007) Applied and Environmental Microbiology 73(13): 4379-4384); U.S. Pat. Nos. 8,420,782 and 8,440,431 and 8,586,526.

An engineered zinc finger or TALE DNA binding domain can have a novel binding specificity, compared to a naturally-occurring zinc finger or TALE protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. As non-limiting examples see U.S. Pat. Nos. 6,453,242 and 6,534,261.

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-PpoI, I-SceII, I-CreI, I-TevI, I-TevII and I-TevIII are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 2007/0117128. DNA-binding domains from meganucleases may also exhibit nuclease activity.

As mentioned above, any nuclease may be operably linked to any at least one additional DNA binding domain. The nuclease may comprise heterologous DNA-binding and cleavage domains (e.g., Cpf1, Cas9, zinc finger nucleases; TALENs, and meganuclease DNA-binding domains with heterologous cleavage domains) or, alternatively, the DNA-binding domain of a naturally-occurring nuclease may be altered to bind to a selected target site (e.g., a meganuclease that has been engineered to bind to site different than the cognate binding site). For example, engineering of homing endonucleases with tailored DNA-binding specificities has been described, see, Chames et al. (2005) Nucleic Acids Res 33(20):e178; Arnould et al. (2006) J. Mol. Biol. 355:443-458 and Grizot et al (2009) Nucleic Acids Res July 7 e publication. In addition, engineering of ZFPs has also been described. See, e.g., U.S. Pat. Nos. 6,534,261; 6,607,882; 6,824,978; 6,979,539; 6,933,113; 7,163,824; and 7,013,219.

In certain embodiments, the nuclease domain is a meganuclease (homing endonuclease) domain. Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-PpoI, I-SceII, I-CreI, I-TevI, I-TevII and I-TevIII. Their recognition sequences are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J.

Mol. Biol. 263:163-180; Argast et al. (1998) J. Mol. Biol. 280:345-353 and the New England Biolabs catalogue. Thus, any meganuclease domain (or functional portion thereof) may be combined with any DNA-binding domain (e.g., ZFP, TALE) to form a nuclease. Furthermore, the nuclease domain may also bind to DNA independent of the DNA-binding domain.

DNA-binding domains from naturally-occurring meganucleases, primarily from the LAGLIDADG family, have been used to promote site-specific genome modification in plants, yeast, Drosophila, mammalian cells and mice, but this approach has been limited to the modification of either homologous genes that conserve the meganuclease recognition sequence (Monet et al. (1999), Biochem. Biophysics. Res. Common. 255: 88-93) or to pre-engineered genomes into which a recognition sequence has been introduced (Route et al. (1994), Mol. Cell. Biol. 14: 8096-106; Chilton et al. (2003), Plant Physiology. 133: 956-65; Puchta et al. (1996), Proc. Natl. Acad. Sci. USA 93: 5055-60; Rong et al. (2002), Genes Dev. 16: 1568-81; Gouble et al. (2006), J. Gene Med. 8(5):616-622). Accordingly, attempts have been made to engineer meganucleases to exhibit novel binding specificity at medically or biotechnologically relevant sites (Porteus et al. (2005), Nat. Biotechnol. 23: 967-73; Sussman et al. (2004), J. Mol. Biol. 342: 31-41; Epinat et al. (2003), Nucleic Acids Res. 31: 2952-62; Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication Nos. 2007/0117128; 2006/0206949; 2006/0153826; 2006/0078552; and 2004/0002092). In addition, naturally-occurring or engineered DNA-binding domains from meganucleases have also been operably linked with a cleavage domain from a heterologous nuclease (e.g., FokI) (also known as mega TALs). In other embodiments, the nuclease is a zinc finger nuclease (ZFN). ZFNs comprise a zinc finger protein that has been engineered to bind to a target site in a gene of choice and cleavage domain or a cleavage half-domain.

As noted above, zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al., (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261.

In any of the nucleases described herein, the nuclease can comprise an engineered TALE DNA-binding domain and a nuclease domain (e.g., endonuclease and/or meganuclease domain), also referred to as TALENs. Methods and compositions for engineering these TALEN proteins for robust, site specific interaction with the target sequence of the user's choosing have been published (see U.S. Pat. No. 8,586,526). In some embodiments, the TALEN comprises a endonuclease (e.g., Fold) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res: 1-13, doi: 10.1093/nar/gkt1224). In addition, the nuclease domain may also exhibit DNA-binding functionality.

In still further embodiments, the nuclease comprises a compact TALEN (cTALEN). These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the TevI nuclease domain (see Beurdeley et al (2013) Nat Comm: 1-8 DOI: 10.1038/ncomms2782). Any TALENs may be used in combination with additional TALENs (e.g., one or more TALENs (cTALENs or FokI-TALENs) with one or more mega-TALs).

As noted above, the cleavage domain may be heterologous to the DNA-binding domain, for example a zinc finger or TALE DNA-binding domain and a cleavage domain from a nuclease or a meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

In some embodiments, a RNA-guided DNA nuclease may be used to cause a DNA break at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In some embodiments, the CRISPR protein (e.g., Cas9) is derived from a type II CRISPR system. The Cas9 protein may be derived from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difjicile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

Thus, a RNA guided DNA nuclease of a Type II CRISPR System, such as a Cas9 protein or modified Cas9 or homolog or ortholog of Cas9, or other RNA guided DNA nucleases belonging to other types of CRISPR systems, such as Cpf1 and its homologs and orthologs, may be used in the RNA compositions of the present invention.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

According to one aspect, a nuclease having two or more nuclease domains may be modified or altered to inactivate all but one of the nuclease domains. Such a modified or altered nuclease is referred to as a nickase, to the extent that the nuclease cuts or nicks only one strand of double stranded DNA.

According to certain aspects of methods of RNA-guided genome regulation described herein, Cas9 may be altered to form a nickase. A Cas9 nickase is provided where either the RuvC nuclease domain or the HNH nuclease domain is inactivated, thereby leaving the remaining nuclease domain active for nuclease activity. In this manner, only one strand of the double stranded DNA is cut or nicked.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 or dCas9 and exhibits reduced or eliminated nuclease activity, or nuclease activity is absent or substantially absent within levels of detection. According to this aspect, nuclease activity for a dCas9 may be undetectable using known assays, i.e. below the level of detection of known assays.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. pyogenes and protein sequences having at least 30%, 40%, 50%, 60%, 70%, 80%, 90%, 95%, 98% or 99% homology thereto and being a DNA binding protein, such as a RNA guided DNA binding protein. According to one aspect, an engineered Cas9-gRNA system is provided which enables RNA-guided genome regulation in cells by tethering transcriptional activation domains to either a nuclease-null Cas9 or to guide RNAs.

In some embodiments, the CAS protein is Cpf1, a putative class 2 CRISPR effector. Cpf1 mediates robust DNA interference with features distinct from Cas9. Cpf1 is a single RNA-guided endonuclease lacking tracrRNA, and it utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (Zetsche et al. Cell. 2015).

Additionally, guide RNAs can be engineered to bind to a target of choice in a genome by commonly known methods known in the art for creating specific RNA sequences. These guide RNAs are designed to guide the Cas9 to any chosen target site.

In some embodiments, a nuclease is fused to a domain capable of specifically binding a recognition motif attached to a RNA-based donor. The binding of such a domain to a recognition motif allows the nuclease, RNA-based donor, and target DNA site to be in close proximity upon double-strand break formation. As mentioned above, several domain—recognition motif pairs are known in the art and any such pair may be used for this purpose. Accordingly, the recognition motif attached to the RNA-based donor may be RNA, DNA, or any ligand capable of being specifically bound by a domain fused to the nuclease.

### Donors

As noted above, the present invention discloses a genome editing method to replace a sequence of a DNA target site with a sequence of an exogenous RNA template (also referred to herein as a “RNA-based donor,” “donor RNA template,” “RNA donor,” or more simply “donor,” or “template”). Genome editing methods may be useful, for example, for correction of a mutant gene or for increased expression of a wild-type gene.

It will be readily apparent that the donor sequence is typically not identical to the genomic sequence where it is placed. A donor sequence may contain a non-homologous sequence, i.e, an insert sequence, flanked by two regions of homology. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest. Importantly, a RNA-based donor template of the present invention may incorporate the sequence information which it encodes into the target genomic DNA site by any mechanism. The RNA-based donor polynucleotide may contain a self-annealing RNA segment at one or both termini of the molecule. Such a self-annealing RNA sequences may be separated by a loop. The self-annealing RNA is capable of forming a structure such as a hairpin, which increases the stability of the RNA-based donor polynucleotide.

The RNA-based donor polynucleotide may contain DNA elements at either the 5′ or 3′ end. The DNA elements may hybridize with portions of the RNA-based donor and/or may self-hybridize to form a terminal hairpin. The RNA-based donor may contain a transcription factor binding site in a DNA element at its terminus. A transcription factor bound to a binding site on the RNA-based donor facilitates entry of the RNA-based donor into the nucleus. The transcription factor may also bind the target DNA site. The transcription factor may bind and activate a regulatory region which controls expression of the gene containing the target DNA site. The RNA-based donor may contain a restriction enzyme binding site in a DNA element at its terminus. The RNA-based donor may be capped at the 5′ end. The RNA donor may contain single-stranded and/or double-stranded portions and can be introduced into a cell in linear or circular form. See, e.g., U.S. Patent Publication Nos. 2010/0047805; 2011/0281361; and 2011/0207221. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A RNA-based donor sequence may be used for gene correction or targeted alteration of an endogenous sequence. The RNA-based donor sequence may be introduced to the cell on a vector, may be electroporated into the cell, or may be introduced via other methods known in the art. The RNA donor sequence can be used to ‘correct’ a mutated sequence in an endogenous gene (e.g., the sickle mutation in beta globin), or may be used to insert sequences with a desired purpose into an endogenous locus.

A RNA-based donor sequence may be one component of a RNA composition. The RNA composition may include additional RNAs such as, but not limited to, guide-RNAs, tracr-RNAs, siRNAs, shRNAs, miRNAs, mRNAs and antisense RNAs. An mRNA of the RNA composition may express a protein, e.g. a RNA-guided DNA nuclease, when delivered to a cell. Moreover, a RNA composition including a RNA-based donor sequence may be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The insert sequence of the RNA-based donor may be inserted or copied into a target site so that its expression is driven by the endogenous promoter at the integration site. However, it will be apparent that the donor RNA may comprise a promoter and/or enhancer sequence, for example a constitutive promoter or an inducible or tissue specific promoter.

The RNA-based donor molecule sequence may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPP1R12c (also known as AAVS1) gene, an albumin gene or a Rosa gene. See, e.g., U.S. Pat. Nos. 7,951,925 and 8,110,379; U.S. Publication Nos. 2008/0159996; 2010/00218264; 2010/0291048; 2012/0017290; 2011/0265198; 2013/0137104; 2013/0122591; 2013/0177983 and 2013/0177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, exogenous RNA sequences may also include transcriptional or translational regulatory sequences, for example, promoters, enhancers, insulators, internal ribosome entry sites, sequences encoding 2A peptides and/or polyadenylation signals.

In certain embodiments, RNA compositions including a RNA-based donor may further comprise a RNA sequences selected from the group consisting of a gene encoding a protein, a regulatory sequence and/or a sequence that encodes a structural nucleic acid such as a siRNA, shRNA, miRNA and antisense RNA.

### Delivery

The RNA compositions, RNA donors, and additional proteins (e.g., ZFPs, TALENs, CRISPR/Cas, transcription factors, restriction enzymes) and/or polynucleotides encoding same described herein may be delivered to a target cell by any suitable means. The target cell may be any type of cell e.g., eukaryotic or prokaryotic, in any environment e.g., isolated or not, maintained in culture, in vitro, ex vivo, in vivo or in planta.

Any suitable viral vector system may be used to deliver RNA compositions. Conventional viral and non-viral based gene transfer methods can be used to introduce nucleic acids and/or donors in cells (e.g., mammalian cells, plant cells, etc.) and target tissues. Such methods can also be used to administer nucleic acids encoding and/or donors to cells in vitro. In certain embodiments, nucleic acids and/or donors are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds.) (1995); and Yu et al., Gene Therapy 1:13-26 (1994).

Methods of non-viral delivery of nucleic acids and/or proteins include electroporation, lipofection, microinjection, biolistics, particle gun acceleration, virosomes, liposomes, immunoliposomes, polycation or lipid:nucleic acid conjugates, artificial virions, and agent-enhanced-uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. (2006) Trends Plant Sci. 11(1):1-4. Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al. (2015) Nat. Biotechnol. 33(1):73-80. See also Coelho et al. (2013) N. Engl. J. Med. 369, 819-829; Judge et al. (2006) Mol. Ther. 13, 494-505; and Basha et al. (2011) Mol. Ther. 19, 2186-2200. In one embodiment, one or more nucleic acids are delivered as RNA. As described herein, RNA components of the composition being delivered to a cell may be attached to each other by direct ligation or via basepairing. Delivery of modified RNAs is also contemplated. Also optional is the use of capped RNAs to increase translational efficiency and/or RNA stability. Generally, methylated-caps are preferred for mRNAs while non-methylated caps are preferred for RNA-based donors.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al (2009) Nature Biotechnology 27(7) p. 643).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. However, a RNA virus is preferred for delivery of the RNA compositions described herein. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g. Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1): 10-20 (1997); Dranoff et al., Hum. Gene Ther. 1:111-2 (1997).

Packaging cells are used to form virus particles that are capable of infecting a host cell. Such cells include 293 cells, which package adenovirus, AAV, and .psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a RNA composition, and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with the nucleases (e.g. ZFNs or TALENs) or nuclease systems (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-.gamma. and TNF-alpha are known (as a non-limiting example see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panB cells), GR-1 (granulocytes), and lad (differentiated antigen presenting cells) (as a non-limiting example see Inaba et al., J. Exp. Med. 176:1693-1702 (1992)). Stem cells that have been modified may also be used in some embodiments.

Notably, any one of the RNA-based donors described herein is suitable for genome editing in post-mitotic cells or any cell which is not actively dividing, e.g., arrested cells, because RNA templated repair does not necessarily require a homologous recombination event to occur. Examples of post-mitotic cells which may be edited using a RNA-based donor or RNA correction template of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### Applications

The disclosed compositions and methods can be used for any application in which it is desired to perform nuclease-mediated genomic modification in any cell type, including clinical applications nuclease-based therapies feasible in a clinical setting as well as agricultural (plant) applications. For example, the RNA-based donors, RNA compositions and methods described herein will improve the therapies such as: ex vivo and in vivo gene disruption (CCR5) in CD34+ cells (see, e.g., U.S. Pat. No. 7,951,925); ex vivo and in vivo gene correction of hemoglobinopathies in CD34+ cells (see, e.g., U.S. Application No. 61/694,693); and/or ex vivo and in vivo gene addition to albumin locus for therapy of lysosomal storage diseases and hemophilias (see, e.g., U.S. Patent Publication Nos. 2014/0017212 and 2013/0177983). Additionally, the disclosed compositions and methods can be used to in the manufacture of a medicament or pharmaceutical composition to treat genetic disease in a patient.

In addition, the methods and compositions described herein can be used to generate model organisms and cell lines, including the generation of stable knock-out cells in any given organism. Accordingly, the methods described herein can be used to generate cell lines with new properties. This includes cell lines used for the production of biologicals like Hamster (CHO) cell lines or cell lines for the production of several AAV serotypes like human HEK 293 cells or insect cells like Sf9 or Sf21 or genomically-modified plants and plant lines.

The methods and RNA compositions of the invention can also be used in the production of transgenic non-human organisms. Transgenic animals can include those developed for disease models, as well as animals with desirable traits. Embryos may be treated using the methods and compositions of the invention to develop transgenic animals. In some embodiments, suitable embryos may include embryos from small mammals (e.g., rodents, rabbits, etc.), companion animals, livestock, and primates. Non-limiting examples of rodents may include mice, rats, hamsters, gerbils, and guinea pigs. Non-limiting examples of companion animals may include cats, dogs, rabbits, hedgehogs, and ferrets. Non-limiting examples of livestock may include horses, goats, sheep, swine, llamas, alpacas, and cattle. Non-limiting examples of primates may include capuchin monkeys, chimpanzees, lemurs, macaques, marmosets, tamarins, spider monkeys, squirrel monkeys, and vervet monkeys. In other embodiments, suitable embryos may include embryos from fish, reptiles, amphibians, or birds. Alternatively, suitable embryos may be insect embryos, for instance, a Drosophila embryo or a mosquito embryo.

Transgenic organisms contemplated by the methods and RNA compositions of this invention also include transgenic plants and seeds. Examples of suitable transgenes for introduction include an exogenous RNA insert sequence that may comprise a sequence encoding one or more functional polypeptides, with or without one or more promoters. The insert sequence may be integrated in the host genome and impart desirable traits to the organism. Such traits in plants include, but are not limited to, herbicide resistance or tolerance; insect resistance or tolerance; disease resistance or tolerance (viral, bacterial, fungal, nematode); stress tolerance and/or resistance, as exemplified by resistance or tolerance to drought, heat, chilling, freezing, excessive moisture, salt stress; oxidative stress; increased yields; food content and makeup; physical appearance; male sterility; drydown; standability; prolificacy; starch quantity and quality; oil quantity and quality; protein quality and quantity; amino acid composition; and the like. Of course, any two or more exogenous nucleic acids of any description, such as those conferring herbicide, insect, disease (viral, bacterial, fungal, nematode) or drought resistance, male sterility, drydown, standability, prolificacy, starch properties, oil quantity and quality, or those increasing yield or nutritional quality may be employed as desired. In certain embodiments, the exogenous nucleic acid sequence comprises a sequence encoding a herbicide resistance protein (e.g., the AAD (aryloxyalkanoatedioxygenase) gene) and/or functional fragments thereof.

### Kits

In another aspect, the invention provides kits that are useful for increasing gene disruption and/or targeted integration following nuclease-mediated cleavage of a cell's genome. The kits typically include one or more RNAs, including a RNA-based donor, useful for inducing gene editing and insertion of sequence at a target site, as well as instructions for introducing the RNAs into the cells.

In certain embodiments, the kits comprise at least one construct with the target gene and a known nuclease capable of cleaving within the target gene. Such kits are useful for optimization of cleavage conditions in a variety of varying host cell types.

The kits typically contain a RNA composition comprising RNA-based donors as described herein as well as instructions for introducing the RNA composition to cells. The kits can also contain cells, buffers for transformation of cells, culture media for cells, and/or buffers for performing assays. Typically, the kits also contain a label which includes any material such as instructions, packaging or advertising leaflet that is attached to or otherwise accompanies the other components of the kit.

### Example 1

### Induction of Cas9-Mediated RNA-Templated Repair

An assay system to validate the induction of RNA-templated repair following Cas9-mediated DSB using RNA components is described below. The effect of transfection conditions and donor concentration on the efficiency of the process was also determined.

Briefly, 7×10⁴ Hek293 cells stably expressing inactive GFP (iGFP-Hek293) were seeded into a well of a 24-well plate. 24 h after plating, cells were transfected with RNA components using 2 ul, 3 ul or 4 ul of Lipofectamine 3000. Transfection efficiency was measured by transfecting the cells with 500 ng mRNA capable of expressing mCherry (Trilink). Thus, a mCherry positive cell indicates positive transfection of a cell. Based on the proportion of cells exhibiting a mCherry signal, the transfection efficiency ranged between 80 and 90%.

Cells were also transfected with 100 ng sgRNA (Synthago), which targeted a Cas9 nuclease to the inactive GFP sequence. Cas9 was provided to the cells in the form of an mRNA (500 ng, Trilink) which is capable of expressing the nuclease (SEQ ID NO: 1). Either 200 ng or 1000 ng of a 312 nt GFP RNA donor (SEQ ID NO: 2) was used to repair the inactive GFP sequence (SEQ ID NO: 3). The GFP RNA donor was synthesized by applicants using in-vitro transcription (IVT). The IVT was performed using RiboMax kit (Promega), and an unmethylated cap analog (ApppG) was included into the reaction at a ratio of 5:1 cap analog to rGTP, respectively. As controls, cell samples that did not contain sgRNA or a RNA donor were included.

Cells were harvested at 72 hr post transfection. Cell suspensions of each sample were then transferred to a FACS compatible tube for measurement of GFP florescent intensity. Flow cytometry was performed on a BD-LSRII (Becton Dickinson) and Analysis was done using FlowJo FACS analysis software. The GFP signal indicates that the correction of the GFP ORF was accomplished using the GFP correction RNA donor. Based on the results of the assay, which are depicted in FIG. 9A-FIG. 9F, both the RNA donor and the gRNA are essential for the desired RNA-templated repair to occur. Furthermore, the efficiency of the process depends on the dose of the donor, which emphasizes that the repair was mediated by the RNA donor.

### Example 2

RNA Mediated Repair of the CASQ2 Gene in hiPSC Derived Cardiomyocytes

hiPSC-CM cells were infected with lentiviroids bearing a 430 bp DNA donor having nearly complete homology to the human CASQ2 gene, with or without U6 promotor (SEQ ID NO: 4 and SEQ ID NO: 5). Five (5) days after infection, cells were transfected with 250 ng Cas9 mRNA, 50 ng CASQ2 gRNA (ACCCCGATCTGAGCATCCTG (SEQ ID NO: 6)) and 100 ng mCherry mRNA which served as transfection efficiency reporter. The experiment include the following treatments (Table 1): 1) Non-treated control cells; 2) CASQ2 donor without U6 promotor, Cas9 and Grna; 3) CASQ2 donor with U6 promotor, Cas9 and gRNA; 4) CASQ2 donor with U6 promotor but no Cas9 and no Grna; 5) Cas9 and gRNA (no DNA donor). 72 h post transfection, genomic DNA was extracted using E.Z.N.A Tissue DNA Kit (Omega D3396). The CASQ2 gene was amplified by PCR and next-generation sequencing analysis was performed.

As seen in Table 1, the number of total sequence reads from each sample ranged from 31552 to 48197. Typical insertion/deletion pattern was observed in all samples that were transfected with Cas9 and guide RNA (Table 1, samples 2,3,5). The frequency of indel events in those samples was in the range of 8.4% to 16.8%. However, HDR events occur at a significant frequency only in sample 5, in which the cells were infected with a viral element containing a U6 promoter that drives the transcription of the donor DNA.

| Sample | | Total | Indel | HDR |
|---|---|---|---|---|
| No. | Sample description | sequences | frequency | frequency |
| 1 | Untreated control | 40256 | 32 (0.1%) | 0 (0.0%) |
| 2 | Donor W/O U6 | 31552 | 5300 (16.8%) | 8 (0.0%) |
| | promoter | | | |
| 3 | Donor + U6 | 37525 | 3150 (8.4%) | 713 (1.9%) |
| | promotor | | | |
| 4 | Donor + U6 W/O | 48197 | 45 (0.1%) | 7 (0.0%) |
| | Cas9 | | | |
| 5 | Cas9 + gRNA only | 38084 | 3592 (9.4%) | 20 (0.1%) |

What is claimed is:

1. A composition comprising a non-naturally occurring RNA molecule which comprises a donor RNA covalently attached to a tracrRNA, which is in turn covalently attached to a guide RNA having homology to an intended DNA target site which contains a PAM recognition sequence.

2. The composition of claim 1, wherein the donor RNA is between two sequences having homology to the intended DNA target site.

3. The composition of claim 1, wherein the donor RNA contains at least one sequence difference relative to the target DNA site sequence, which at least one sequence difference is an alteration intended to be introduced into the target DNA site sequence.

4. The composition of claim 3, wherein the at least one sequence difference is:
a. a nucleotide or multiple nucleotides in the donor RNA each of which is non-homologous or non-complementary to a corresponding nucleotide or multiple nucleotides of the target DNA site sequence;
b. a nucleotide or multiple nucleotides in the donor RNA which do not have a corresponding nucleotide or multiple nucleotides in the target DNA site sequence;
c. an absence of a nucleotide or multiple nucleotides in the donor RNA which correspond to a nucleotide or multiple nucleotides that are present in the target DNA site sequence; or
d. any combination of the above.

5. The composition of claim 1, wherein a linker connects the donor RNA to the tracrRNA

6. The composition of claim 1, wherein the DNA target site is in eukaryotic genomic DNA.

7. The composition of claim 1, further comprising at least one mRNA encoding an RNA-guided DNA nuclease.

8. The composition of claim 1, further comprising at least one RNA-guided DNA nuclease.

9. The composition of claim 8, wherein the RNA-guided DNA nuclease is a nickase and the RNA donor targets the same DNA strand that is targeted by the guide RNA.

10. A vector encoding the non-naturally occurring RNA molecule of claim 1.

11. A host cell containing the composition of claim 1.

12. The host cell of claim 11, wherein the cell is selected from the group consisting of a myocyte, a cardiomyocyte, a hepatocyte, an osteocyte and a neuron.

13. The host cell of claim 11, wherein the cell is a eukaryotic cell.

14. The host cell of claim 13, wherein the cell is a mammalian cell or a plant cell.

15. The host cell of claim 11, wherein the cell is in culture.

16. A method of genome editing in a cell comprising delivering to a cell the composition of claim 1.

17. The method of claim 16, wherein the delivery method is selected from the group consisting of electroporation, lipofection, microinjection, biolistics, particle gun acceleration, cationic-lipid mediated delivery and viral mediated delivery.

18. The method of claim 16, wherein the delivery is selected from the group consisting of in vivo, in vitro and ex vivo delivery.

19. A non-human transgenic organism formed by the method of claim 16.

20. A kit comprising the composition of claim 1 and instructions for use thereof.
